(19) United States
(12) Patent Application Publication
(10) Pub. No.: US 2024/0120101 A1
(43) Pub. Date: Apr. 11, 2024

(54) DISEASE CLASSIFIER AND DYSBIOSIS INDEX TOOLS

(71) Applicant: SHEBA IMPACT LTD., Ramat Gan (IL)

(72) Inventors: Yael HABERMAN ZIV, Tel Aviv (IL); Haya ABBAS, Kfar Kana (IL); Tzipi BRAUN, Giv'at Shmuel (IL); Amnon AMIR, Herzliya (IL)

(21) Appl. No.: 18/509,835

(22) Filed: Nov. 15, 2023

Related U.S. Application Data
(63) Continuation of application No. PCT/IL2022/050521, filed on May 19, 2022.
(60) Provisional application No. 63/191,006, filed on May 20, 2021.

(51) Int. Cl. G16H 50/20; G16H 20/10

(52) U.S. Cl. CPC G16H 50/20; G16H 20/10

(57) ABSTRACT

Herein disclosed are a computer-implemented method, a system, and a kit for assessing/classifying the health status associated with a microbiome profile of a gastrointestinal (GI) sample. The method includes receiving data regarding expression levels of amplicon sequence variants (ASVs) of a V4 region of 16S rRNA in a GI sample of a subject, and utilizing a machine learning algorithm that is trained to distinguish a healthy state from a sick state, a score is computed, and a prediction is made for the presence of a general microbial response that is shared by a large variety of diseases.

### CROSS REFERENCE TO RELATED APPLICATIONS

This application is a Bypass Continuation of PCT Patent Application No. PCT/IL2022/050521 having International filing date of May 19, 2022, which claims the benefit of priority of U.S. Provisional Patent Application No. 63/191,006, filed May 20, 2021, the contents of which are all incorporated herein by reference in their entirety.

### REFERENCE TO AN ELECTRONIC SEQUENCE LISTING

The contents of the electronic sequence listing (TSH_015-WOUS_Seq_Listing.xml; Size: 151,894 bytes; and Date of Creation: Dec. 17, 2023) is herein incorporated by reference in its entirety.

### FIELD OF THE INVENTION

The present disclosure is related to the field of gut microbiome association with human health and disease states. The disclosure provides a computer method, a system, and a kit for assessing/classifying health status.

### BACKGROUND OF THE INVENTION

Despite numerous studies linking the microbiome to human health and disease conditions, there are still many gaps regarding gut bacterial compositions and profiles and how those contribute to various human pathogenesis.

For example, many studies have observed structural imbalances or dysbiosis in the gut microbial composition of Inflammatory bowel diseases (IBD) patients with decreased biodiversity, lower abundance of Firmicutes, and an increased abundance of Gammaproteobacteria. It is therefore anticipated that alteration in the gut microbiome likely plays a central role in IBD pathogenesis, resulting in abnormal interactions between gut microbes, gut epithelia, and the intestinal immune system.

Nevertheless, consistent individual microbial alterations in IBD were not reported and the precise mechanisms of intestinal microbiota dysfunction in IBD remain to be elucidated.

In addition, many other disease-control studies, most of which are not linked with chronic gut inflammation, have shown alterations in the gut microbial composition. Shifts in microbial profiles were observed in various diseases, spanning various conditions, including neuro-psychiatric, metabolic, and malignant diseases.

There is, therefore, an unmet need to characterize host interactions with the gut microbiota distinguishing health from a disease state.

### SUMMARY OF INVENTION

According to some aspects, the present disclosure provides a computer-implemented method and a system for assessing/classifying the health status associated with a microbiome profile of a gastrointestinal (GI) sample. The method utilizes a machine learning algorithm that is trained to distinguish a healthy state from a sick state, and to compute a score that includes a prediction of the presence of a general microbial response in the sample that is shared by a large variety of diseases.

In some embodiments, the method assesses the health status by identifying the level of dysbiosis. In some embodiments, the method assesses the health status by classifying the disease status. In some embodiments, the method prioritizes the health status associated with a microbiome profile for selecting a donor sample having low disease probability for fecal microbiota transplantation (FMT).

According to some aspects, there is disclosed a kit for identifying non-specific amplicon sequence variants (ASVs) of the V4 region of 16S rRNA in a GI sample of a subject.

According to some aspects, there is provided a computer-implemented method for assessing/classifying a health status associated microbiome profile of a gastrointestinal (GI) sample, the method comprising:
receive data regarding an expression level of at least about 40 non-specific amplicon sequence variants (ASVs) of a V4 region of 16S rRNA in a GI sample of a subject obtaining a GI sample of a subject;
analyzing the expression level of at least about 40 non-specific amplicon sequence variants (ASVs) of the V4 region of 16S rRNA;
computing a dysbiosis index (DI) score for the GI sample by applying a trained machine learning algorithm on the determined/received expression level of the at least about 40 ASVs, wherein an altered expression of at least a portion of the at least about 40 ASVs is shared by a plurality of diseases, and wherein at least 5 of the non-specific ASVs have a sequence as set forth in any one of SEQ ID NO: 1-42;
assessing/classifying the health status associated microbiome profile of the subject based on the DI score.

According to some aspects, there is provided a computer-implemented method for assessing/classifying a health status associated microbiome profile of a gastrointestinal (GI) sample.

According to some embodiments, the method comprises receiving data regarding an expression level of at least about 40 non-specific amplicon sequence variants (ASVs) of a V4 region of 16S rRNA in a GI sample of a subject.

According to some embodiments, the method comprises receiving data regarding an expression level of at least 5 ASVs, at least 10 ASVs, at least 20 ASVs, at least 30 ASVs, at least 40 ASVs, at least 50 ASVs, at least 60 ASVs, at least 70 ASVs, at least 80 ASVs, at least 90 ASVs, at least 100 ASVs, at least 110 ASVs, at least 120 ASVs. Each possibility is a different embodiment.

Advantageously, the method comprises computing a dysbiosis index (DI) score for the GI sample by applying a trained machine learning algorithm on the expression level of the at least about 40 ASVs.

Advantageously, an altered expression of at least a portion of the at least about 40 ASVs is shared by a plurality of diseases, and at least 5 of the non-specific ASVs have a sequence as set forth in any one of SEQ ID NO: 1-42.

According to some embodiments, the method comprises assessing/classifying the health status associated microbiome profile of a subject based on the DI score.

According to some embodiments, the machine learning algorithm is trained on a data set comprising the expression level of the at least about 40 ASVs of a large plurality of GI samples obtained from subjects suffering from one or more of the plurality of diseases and from healthy subjects, and a plurality of labels associated with the large plurality of GI samples, each label indicating the health status of the GI sample.

According to some embodiments, the changed expression of ASVs of the V4 region of 16S rRNA comprises comparing the GI sample to control samples.

According to some embodiments, the changed expression of the at least portion of the at least about 40 ASVs comprises upregulation in the expression of about 15 ASVs and downregulation in the expression of about 5 ASVs.

According to some embodiments, the changed expression of the at least portion of the at least about 40 ASVs further comprises upregulation in the expression of about 30 ASVs and downregulation in the expression of about 10 ASVs.

According to some embodiments, assessing a health status associated microbiome profile of a gastrointestinal (GI) sample comprises identifying dysbiosis in a GI sample.

According to some embodiments, identifying dysbiosis in a GI sample comprises prioritizing the degree of dysbiosis.

According to some embodiments, prioritizing the degree of dysbiosis in a GI sample comprises computing a dysbiosis index (DI) score.

According to some embodiments, the machine learning algorithm comprises a classification algorithm.

According to some embodiments, the classifier implements disease status classification of the GI sample to a plurality of disease categories selected from inflammatory diseases, autoimmune diseases, infectious diseases, psychiatric diseases, neurological disorders, metabolic diseases, inflammatory bowel diseases, malignancies, and any combination thereof.

According to some embodiments, the classifier further implements disease status classification of the GI sample to a plurality of diseases selected from a group of GI diseases consisting of: crohn's disease, ulcerative colitis, inflammatory bowel disease, irritable bowel disease, gastroenteritis, clostridioides difficile infection, and cancer, and/or selected from a group of non-GI diseases consisting of: Alzheimer, anorexia, autism, bipolar, depression, chronic fatigue syndrome, diabetes T1, diabetes T2, gout, heart disease, HIV, hepatitis, hypertension, lupus, obesity, pancreatitis, rheumatoid arthritis, schizophrenia, parkinson's disease, and psoriasis.

According to some embodiments, the method further comprises identifying one or more etiologies associated with the GI sample.

According to some embodiments, the method further comprises classifying the subject as suffering from one/or more diseases based on the identified one or more etiologies associated with the GI sample.

According to some embodiments, identifying the one or more etiologies associated with the GI sample comprises further analyzing the expression level of disease-specific ASVs of the V4 region of 16S rRNA.

According to some embodiments, the analysis of altered expression of disease-specific ASVs comprises about 15 IBD-specific ASVs.

According to some embodiments, altered expression of at least a portion of the IBD-specific ASVs is shared by Ulcerative colitis (UC)- and Crohn's disease (CD) and at least 3 of the IBD-specific ASVs have a sequence as set forth in any one of SEQ ID NO: 129-143.

According to some embodiments, changed expression of the about 15 IBD-specific ASVs comprises upregulation in the expression of about 13 ASVs and downregulation in the expression of about 2 ASVs.

According to some embodiments, the one or more etiologies related to the IBD-specific ASVs are selected from Ulcerative colitis (UC) and Crohn's disease (CD).

Advantageously, the method further comprising the step of prioritizing and/or selecting a donor sample for fecal microbiota transplantation (FMT), wherein the sample has a low disease probability.

According to some aspects, there is disclosed a system for assessing/classifying a health status associated microbiome profile of a gastrointestinal (GI) sample, the system comprises a processing logic configured to:
receive data regarding the expression level of at least 40 non-specific amplicon sequence variants (ASVs) of the V4 region of 16S rRNA in a GI sample of a subject;
computing a dysbiosis index (DI) score for the GI sample by applying a trained machine learning algorithm on the determined/received expression level of at least about 40 ASVs, wherein an altered expression of at least a portion of the at least about 40 ASVs is shared by a plurality of diseases and wherein at least 5 of the ASVs have a sequence as set forth in any one of SEQ ID NO: 1-42;
assessing/classifying the health status associated microbiome profile of the subject based on the DI score.

According to some aspects, there is disclosed a system for assessing/classifying a health status associated microbiome profile of a gastrointestinal (GI) sample, the system comprises a processing logic.

According to some embodiments, the processing logic is configured to receive data regarding the expression level of at least 40 non-specific amplicon sequence variants (ASVs) of the V4 region of 16S rRNA in a GI sample of a subject.

According to some embodiments, the processing logic is configured to compute a dysbiosis index (DI) score for the GI sample by applying a trained machine learning algorithm on the determined/received expression level of at least about 40 ASVs.

According to some embodiments, an altered expression of at least a portion of the at least about 40 ASVs is shared by a plurality of diseases and at least 5 of the ASVs have a sequence as set forth in any one of SEQ ID NO: 1-42;

According to some embodiments, the processing logic is configured to assessing/classifying the health status associated microbiome profile of the subject based on the DI score.

According to some aspects, there is disclosed a kit comprising: primers capable of identifying at least about 40 non-specific amplicon sequence variants (ASVs) of the V4 region of 16S rRNA in a GI sample of a subject.

According to some embodiments, the kit comprises primers capable of identifying at least 5, at least 10, at least 15, at least 20, at least 30, at least 40, at least 50, at least 60, at least 70, at least 80, at least 90, at least 100, at least 120, or all of the non-specific amplicon sequence variants (ASVs) of the V4 region of 16S rRNA in a GI sample of a subject. Each possibility is a different embodiment.

According to some embodiments, at least 5 of the non-specific ASVs identified by the primers are selected from SEQ ID NO: 1-42.

According to some embodiments, the primers comprise at least a single pair of primers and wherein each pair can identify at least a single ASV.

According to some embodiments, the kit comprises at least a single pair of primers, at least 2 pairs of primers, at least 3 pairs of primers, at least 5 pairs of primers, at least 10 pairs, at least 20 pairs of primers. Each possibility is a different embodiment.

According to some embodiments, each pair of primers can identify at least a single ASV, at least 2 ASVs, at least 5 ASVs, at least 10 ASVs, at least 20 ASVs, at least 30 ASVs, at least 40 ASVs, at least 50 ASVs, at least 60 ASVs, at least 70 ASVs, at least 80 ASVs, at least 90 ASVs, at least 100 ASVs, at least 110 ASVs, at least 120 ASVs. Each possibility is a different embodiment.

According to some embodiments, the kit further comprises equipment for collecting, storing, and labeling a subject's GI sample.

According to some aspects, there is provided a method for evaluating a GI sample, the method comprising:
obtaining a GI sample of a subject;
analyzing an expression level of at least about 40 non-specific amplicon sequence variants (ASVs) of the V4 region of 16S rRNA;
computing a dysbiosis index (DI) score for the GI sample by applying a trained machine learning algorithm on the determined/received expression level of the at least about 40 ASVs, wherein an altered expression of at least a portion of the at least about 40 ASVs is shared by a plurality of diseases, and wherein at least 5 of the non-specific ASVs have a sequence as set forth in any one of SEQ ID NO: 1-42;
assessing/classifying the health status associated microbiome profile of the GI sample based on the DI score.

According to some embodiments, the method comprises analyzing the expression level of at least 5 ASVs, at least 10 ASVs, at least 20 ASVs, at least 30 ASVs, at least 40 ASVs, at least 50 ASVs, at least 60 ASVs, at least 70 ASVs, at least 80 ASVs, at least 90 ASVs, at least 100 ASVs, at least 110 ASVs, at least 120 ASVs. Each possibility is a different embodiment.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described in relation to certain examples and embodiments with reference to the following illustrative figures so that it may be more fully understood.

FIGS. 1A-1D presents analyses showing that the pipeline used for microbial characterization, overcomes cohort-specific determinants and enables comparisons between disease-cohorts.

12,838 human gut samples, spanning 59 disease-cohorts comparisons linked with 28 unique diseases and 34 different original studies. V4 16S amplicon sequencing raw reads were processed separately for each original study using Deblur, resulting in bacterial amplicon sequence variants (ASVs). To evaluate the process before and after applying the effect size within each disease-cohort comparison, a subset of up to randomly chosen 23 controls and 23 cases were used from each disease-cohort to avoid sample size bias, resulting in 2356 samples.

FIG. 1A presents Bray Curtis PERMANOVA variance test that was applied to quantify the contribution of different factors affecting the gut microbial composition before the effect size was applied within each disease-cohort comparison. The ASVs of all 2356 samples were included (leftmost column), or only the means obtained in the control and disease groups in each of the 59 disease-cohort (see methods) together for the 59 disease states and controls, and separately for the 59 controls and 59 diseases as indicated. PERMANOVA shows that the original study as well as the specific disease-cohort explains most (44%) of the variation if the effect size within each disease-cohort comparison is not considered. (*=P≤0.01). Total n is shown in brackets.

FIG. 1B presents a Histograms for Bray-Curtis distances between case and control pairs from the same cohort (left), control sample pairs from different cohorts (middle), and case sample pairs from different cohorts (right) indicating higher similarities between disease and controls from the same cohort in comparison to controls from a different cohort, emphasizing the original cohort contribution.

FIGS. 1C-1D presents a scatter plot of PCoA analysis showing Bray Curtis distances for the mean relative abundance of ASVs in each disease-cohort group, before (FIG. 1C) and after (FIG. 1D) applying the effect size within each disease-cohort comparison. The plot is colored by the original studies or disease-cohorts included in the analysis.

FIGS. 2A-2B. illustrate the process of the meta-analysis used to define general and specific disease signatures.

FIG. 2A illustrates the per-study effect size-based pipeline for the reanalysis of case/control raw reads. 12,838 human gut samples, spanning 59 disease cohorts linked with 28 unique diseases (a). Per-sample V4 16S amplicon sequencing raw reads were processed using Deblur, resulting in bacterial amplicon sequence variants (ASVs) (b). Potential disease dependent ASVs within each original study were identified separately within each disease cohort (rank mean tests, FDR<0.25, using a random subset of 23 cases and 10 controls to include as many case/control comparison and avoid dominance of large cohort size) (c). ASVs were then combined, resulting in 731 unique ASVs (d). For each disease cohort, the effect size (normalized rank mean difference—NRMD) was calculated for these 731 ASVs using all available samples in each cohort (n=12,838) (e).

FIG. 2B illustrates the process of defining disease signatures based on 731 unique ASVs. The results were combined into a single heatmap table (a). Each column in the heatmap represents a single disease cohort, and each row represents a single ASV, with color representing the NRMD effect size; red and blue colors indicate higher or lower in disease respectively, while white indicates ASVs not present in the disease cohort. Non-disease specific ASVs were identified using a binomial test (FDR<0.1) (b), whereas CD/UC specific ASVs were identified using rank-mean test (FDR<0.1) (c).

FIGS. 3A-3C present analyses of the similarities and differences in the microbial composition between diseases.

FIG. 3A presents a Modified Bray-Curtis distance matrix as a heatmap that was calculated using the 731 ASV effect size ratios between cases and controls across the different disease cohorts using all samples (n=12,838). Comparisons were performed between two disease cohorts only on ASVs found in both. This modified Bray-Curtis metric was used to build the distance matrix ( ). Darker color indicates high similarity and bright color indicates low similarity. Bar colors on X and Y axes indicate the specific disease of each disease cohort, as indicated in the disease key. CD UC and IBD are all colored in dark-red but the labeling specifically indicates the disease type; however, those tend to intermix. IBD represent studies in which patients were only labeled as IBD rather than CD or UC.

FIG. 3B-3C present a principal coordinate analysis (PCoA) plot generated based on the distance matrix depicting disease cohort similarity, where disease cohorts are colored by country (FIG. 3B) or specific disease (FIG. 3C).

FIGS. 4A-4D show analyses of non-specific microbial signal shared across diseases.

FIG. 4A presents a heatmap showing 128 non-specific ASVs identified by applying a binomial test on the ratios of the 731 ASVs across all diseases (FDR<0.1). Columns are disease cohorts, and rows represent the non-specific ASVs that were significantly changed in at least four different diseases, with colors representing the case-control log ratio. Red and blue indicate higher or lower abundance in disease, respectively, while white indicates ASVs not present in the study.

FIG. 4B shows a stacked column chart of non-specific ASVs bacteria that were separated to two groups; those lower in disease (76% of non-specific ASVs, left column), and those that are higher in disease (24% of non-specific ASVs, right column). For those groups, ASV's taxonomic classification was defined, and results are shown in three taxonomic levels as indicated: phylum, order, and class.

FIG. 4C presents functional analysis that was performed using per-ASV predictions of KEGG ontologies via PICRUSt2, to infer microbial community functions significantly enriched in non-specific ASVs showing an increase (top) or decrease (bottom) across different diseases.

FIG. 4D presents a word cloud that was generated via dbBact (http://dbbact.org/) using the disease-increasing non-specific bacteria, indicating that non-specific disease-increasing non-specific bacteria have been previously observed in fecal human adults' samples.

FIGS. 5A-5B present analyses of Ulcerative colitis and Crohn's disease specific microbial signal.

FIG. 5A present a heatmap showing 15 CD/UC “specific” ASVs with significantly higher (or lower) effect size in fecal samples of CD and UC cases compared to controls in comparison to other disease cohorts [rank-mean test on the effect sizes in 10 fecal CD and UC studies vs. other disease (n=45)]. Columns are disease cohorts, and rows represent the CD/UC specific ASVs with colors representing the effect size; red indicates higher abundance and blue indicates lower abundance in cases vs. controls, and white indicates ASVs not present in the study. FIG. 5B A word cloud was generated using dbBact (http://dbbact.org/) using the increasing UC/CD-specific bacteria, indicating that UC/CD-specific increased bacteria has been previously found in fecal and saliva human samples.

FIGS. 6A-6C present disease classifier dysbiosis index that predicts general microbial signal.

FIG. 6A presents a Random Forest classifier heatmap, showing the disease/control prediction AUC in each disease cohort (columns) based on training the classifier on datasets in the different rows (see methods for details). Blue indicates high prediction AUC and red indicates AUC<0.5. Training and prediction in each comparison were performed only on shared ASVs between the trained and the predicted cohorts. Marked Squares in the heatmap, indicate the prediction results obtained after training of the classifier using the same cohort.

FIG. 6B presents a Random Forest classifier heatmap, showing the prediction AUC after performing random permutation of labels of the predicting cohort prior to the classifier prediction, to further validate the non-random results obtained in A.

FIG. 6C presents a dysbiosis index per dataset that was measured by two models: per-sample rank, and by using Gevers CD dysbiosis index, and the resulting P-value (Mann-Whitney) for each dataset is shown in the plot where the x-axis showing the sample-rank model (NSDI), and the y-axis showing the Gevers model. (CD dysbiosis index) ** Diseases with 2 cohorts or more were colored by a specific color, while single cohorts were all colored in gray.

FIGS. 7A-7B present heatmaps showing disease classification that predicts control and disease samples when shuffling the samples before prediction.

FIG. 7A presents a Random Forest classifier heatmap, showing the prediction accuracy after performing random permutation of labels of the predicting cohort prior to the classifier prediction, to further validate the non-random results obtained in FIG. 6A.

FIG. 7B presents a Random Forest classifier heatmap, showing the prediction accuracy after performing random permutation of labels of the training cohort prior to the classifier prediction, to further validate the non-random results obtained in FIG. 6A.

Red indicates high prediction AUC and blue indicates AUC<0.5. Training and prediction in each comparison were performed only on shared ASVs between the trained and the predicted cohorts. Marked squares in the heatmap, indicate the prediction results obtained after training of the classifier using the same cohort.

FIG. 8 presents the same dysbiosis index as shown in FIG. 6C. Each disease-cohort is marked with a reference number.

FIG. 9 presents dysbiosis index that was generated by the per-sample rank method showing more significant values between case/control samples. Dysbiosis index per dataset was measured by two models: per-sample rank, and by using Gevers CD dysbiosis index, and the resulting P-value (Mann-Whitney) for each dataset is shown in the plot where the x-axis showing p-value, and the y-axis showing the number of cohorts.

FIGS. 10A-10C present the performance of the dysbiosis index in other independent disease cohorts. Cohorts were processed in Sheba hospital and NSDI was calculated. The first cohort is of 75 lung cancer and 31 controls. The other is from 32 controls 12 with recent accidents (past 3 months) of hospitalization in the rehab unit and another 19 that are more than 6 months from the accident, under ambulatory rehab clinical follow up.

FIG. 10A presents a box plot showing Faith alpha diversity in cancer and control groups. Mann-Whitney test. *<0.05, **<0.01, ***<0.001.

FIG. 10B presents a box plot showing Faith NSDI in cancer and control groups. Shown in comparison to FIG. 10A. Mann-Whitney test. *<0.05, **<0.01, ***<0.001.

FIG. 10C presents a box plot showing NSDI in hospitalized and discharged rehab cases and control groups. Mann-Whitney test. *<0.05, **<0.01, ***<0.001.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

The term “health status associated microbiome profile” refers to the bacterial composition in a gastrointestinal (GI) sample associated with a certain health state. As used herein, the term indicates that the gut bacterial composition reflects the health state of the host/subject.

In some embodiments, analyzing the expression of bacterial amplicon sequence variants (ASVs) in a GI sample identifies gut microbial composition associated with a certain health state.

As used herein, the terms, “microbiome profile” and “bacterial composition” refer to the microbial composition in the gut and may be interchangeably used.

As used herein, the term “subject” refers to a donor or a patient who provided a GI sample. As used herein, the term “host” refers to a subject who harbors a gut microbiome. The terms “subject” and “host” may be used interchangeably.

As used herein, the term “health status” refers to a healthy state or a sick state, as would be considered according to a common medical diagnosis method. The term “health status” may also include a spectrum (range) of prioritized states in between healthy and sick. In some embodiments, the term “health status” may also refer to the classification of a GI sample, a subject, and/or a microbiome profile to disease categories/status/etiologies (i.e., “disease status”). The terms, “health status” and “health state” may be interchangeably used.

In some embodiments, the term health state may be used in reference to the GI sample. In some embodiments, the term health state may be used in reference to the subject. In some embodiments, the health status may be used in reference to the microbiome profile associated with a GI sample or a subject. In some embodiments, the health status may be used in reference to a sick state or a healthy state. In some embodiments, the health status may be used in reference to identification of dysbiosis. In some embodiments, the health status may be used in reference to a range of prioritized states in between sick and healthy. In some embodiments, the health status may be used in reference to a disease status.

As used herein, the term “disease status” refers to the classification of a GI sample, a subject and/or a microbiome profile to clinically defined disease categories/status/etiologies as would be considered according to a common medical diagnosis method.

The terms, “disease status” and “disease state” may be interchangeably used, unless otherwise stated or otherwise evident from the context. For example, in some embodiments, the term “disease state” may refer to a state of sickness as within the meaning of the herein abovementioned “sick state”).

According to some embodiments of the present disclosure, the health state is determined according to a dysbiosis index (DI) score given to a GI sample. In some embodiments, the health state may be a healthy state or a sick state. In some embodiments, the health state may include identifying and/or prioritizing the degree of dysbiosis in a GI sample. In some embodiments the health status includes classification to disease categories, status, and ethnologies.

As used herein, the term “amplicon sequence variants (ASVs)” or “bacterial ASVs” refers to a DNA segment or a DNA sequence of the V4 region of 16S rRNA. The sequence of the ASVs of the V4 region of 16S rRNA may be determined by RNA sequencing or any other method known in the art for the determination of nucleic acid sequences. As used herein, the terms “ASVs” and “bacterial ASVs” may be used interchangeably.

In some embodiments, determining bacterial ASVs in a GI sample allows family and/or genus level microbiome profiling, and ASV-based taxonomic classification. In some embodiments, 731 bacterial ASVs were associated with the 28 different diseases and served as an input for the machine learning.

As used herein, the term “dysbiosis index score” (“DI score”) refers to a measure that describes the level of compatibility between a microbiome profile of a GI sample and a respective health state represented/predicted by the index. The DI score is used to differentiate between healthy controls and disease cases across a plurality of diseases, thereby assessing/classifying the health status associated microbiome profile.

According to some embodiments of the present disclosure, the DI index may be non-specific (NSDI). Accordingly, the terms “dysbiosis index (DI) score” and “non-specific dysbiosis index (NSDI) score” may be interchangeably used.

The term, “plurality of diseases” refers to at least 3 diseases, at least 5 diseases, at least 10 diseases, at least 15 diseases, at least 20 diseases, or at least 25 diseases. Each possibility is a separate embodiment.

The term “non-specific dysbiosis index” (“NSDI”) refers to a DI that was formulated to include altered expression of ASVs shared between a plurality of diseases thereby representing a general response in the microbiome composition. Therefore, due to its non-specific microbial response/signal associated with non-specific ASVs, the non-specific index predicts a general host state of dysbiosis, which is shared between a plurality of diseases.

As used herein, the term, “non-specific amplicon sequence variants (non-specific ASVs)” refers to a group/signature of a subset of ASVs sharing an altered expression in a plurality of diseases. The non-specific ASVs are associated with a general response in the microbiome composition which is shared between a plurality of diseases.

Accordingly, alterations in ASVs expression profile may be indicative of changes in the level of expression or level of abundance of RNA transcripts having the sequence of the V4 region of 16S rRNA and/or indicative of changes in associated abundance of bacterial taxa (i.e., microbial composition).

According to some embodiments of the present disclosure, a NSDI score may be computed based on the expression of at least a portion of the non-specific ASVs. In some embodiments, changes in the expression of at least a portion of these disease non-specific ASVs are utilized as a marker/signature/signal for dysbiosis. As used herein, the terms “non-specific ASVs”, “disease non-specific ASVs”, “shared ASVs”, “non-specific signal”, and “non-specific response” may be interchangeably used.

According to some embodiments, the NSDI may include only ASVs that have an altered expression in at least 2, at least 3, at least 4, or at least 5 diseases. Each possibility is a separate embodiment. According to some embodiments, the NSDI may be devoid of ASVs the altered expression of which is associated with two types of disease or with a single disease.

As used herein, the term “portion” may refer to about 5 ASVs, about 10 ASVs, about 15 ASVs, about 20 ASVs, about 30 ASVs, about 40 ASVs, about 50 ASVs, about 60 ASVs, about 70 ASVs, about 80 ASVs, about 90 ASVs, about 100 ASVs, or about 120 ASVs.

The term, “specific amplicon sequence variants” (“specific ASVs”) refers to a group/signature of a subset of ASVs sharing an altered expression that is exclusive to a certain disease. The specific ASVs are associated with a characteristic response in the microbiome composition which is unique to a certain disease. As used herein, the terms “specific ASVs” or “disease-specific ASVs” may be interchangeably used.

According to some embodiments, a signature of specific ASVs may be exclusively associated with less than 5 diseases, less than 4 diseases, less than 3 diseases, less than 2 diseases, or exclusive to a single disease only.

As used herein, the terms “about” or “approximately” in reference to a number are generally taken to include numbers that fall within a range of 15% or in the range of 5% in either direction (greater than or less than the number) unless otherwise stated or evident from the context.

The term (disease) “classification algorithm” or (disease) “classifier” refers to an algorithm that implements classification by mapping input data to categories. As used herein, a disease classification algorithm or disease classifier refers to an algorithm that implements the classification to disease categories/status/etiologies by mapping input data comprising non-specific ASVs expression levels in a GI sample

In some embodiments, non-limiting examples of classification algorithms include linear classifiers, quadratic classifiers, kernel estimation, decisions trees (e.g., random forests), neural networks and genetic programing. Each possibility is a separate embodiment.

In some embodiments, a classifier implements disease status classification of the GI sample to a plurality of disease categories selected from inflammatory diseases, autoimmune diseases, infectious diseases, psychiatric diseases, neurological disorders, metabolic diseases, inflammatory bowel diseases, malignancies, and any combination thereof. Each possibility is a separate embodiment.

In some embodiments, a classifier implements disease status classification of the GI sample to GI diseases and/or non-GI disease

In some embodiments, a classifier implements disease status classification of the GI sample to a plurality of disease categories selected from the group consisting of GI diseases: crohn's disease, ulcerative colitis, inflammatory bowel disease, irritable bowel disease, gastroenteritis, clostridioides difficile infection, and cancer, and/or selected from the group consisting of non-GI diseases: Alzheimer, anorexia, autism, bipolar, depression, chronic fatigue syndrome, diabetes T1, diabetes T2, gout, heart disease, HIV, hepatitis, hypertension, lupus, obesity, pancreatitis, rheumatoid arthritis, schizophrenia, parkinson's disease, and psoriasis. Each possibility is a separate embodiment.

In some embodiments, a classifier implements disease status classification of the GI sample to a plurality of etiologies.

The term “gastrointestinal (GI) sample” may refer to a biopsy and/or stool/fecal samples. As used herein, in some embodiments, a gastrointestinal biopsy includes but is not limited to samples collected from the terminal ileum, rectum, small and large intestine.

The term “obtaining a GI sample of a subject” may refer to receiving a test tube containing a GI sample associated with a subject. In some embodiments, the step of obtaining a GI sample may be performed by a third party that collects the sample from the subject and may store it or transfer it for storage, until further analysis is performed on the sample.

The term “analyzing the expression level” may mean that in some embodiments, the step of analyzing the expression of the ASVs associated with a GI sample may also be performed by same or different third party that collected and/or stored the sample.

The term “receiving data regarding the expression level” may mean that in some embodiments, the step of receiving data regarding the expression of the ASVs associated with a GI sample may mean receiving data from same or different third party that collected and/or stored, and/or analyzed the sample.

The term “computing a dysbiosis index (DI) score” may mean that in some embodiments, the step of computing a DI score is performed following obtaining the sample by a third party, and/or analyzing ASVs expression by same or different third party, and/or receiving the data from same or different third party. In some embodiments, the determined expression level of the ASVs associated with the GI sample is received and the data is entered as input into the machine learning algorithm utilized for computing a dysbiosis index (DI) score for the GI sample.

The term, “equipment for collecting, storing, and labeling a subject's GI sample”, refers to various means that can be utilized for GI sample collection, storage, and identification including but not limited to a ladle, a container, and a labeling sticker, or same.

As used herein, the singular forms “a”, “an” and “the” are intended to include the plural forms as well, unless the context clearly indicates otherwise.

According to some embodiments, the V4 regions are used to specifically compare the specific ASV rather than the taxonomy level and to calculate the per ASV effect size within original cohorts between cases and controls, which also minimize the study-specific signature.

According to some embodiments, the herein disclosed robust non-specific general response is dominated by reduction of microbial ASVs (97 ASVs having sequences set forth in any one of SEQ ID NO: 31-42 and SEQ ID NO: 44-128), with a smaller group of bacterial ASVs (31 ASVs having sequences set forth in any one of SEQ ID NO: 1-30 and SEQ ID NO: 43) that were up-regulated in a large array of diseases.

According to some embodiments, the ASVs included in the NSDI include at least 5, at least 10, at least 20, at least 40, at least 80, at least 120, or all of the ASVs listed in SEQ ID NO: 1-128. Each possibility is a separate embodiment.

According to some embodiments, the ASVs included in the NSDI include at least 5, at least 10, at least 15, at least 20, at least 30, at least 40, or all of the ASVs listed in SEQ ID NO: 1-42. Each possibility is a separate embodiment.

According to some embodiments, these non-specific microbial changes reflect a general response of the body to pathologic conditions.

According to some embodiments, disease classifiers performed well in identifying many sick vs. healthy states, likely due to the general non-specific signal.

According to some embodiments, most identified IBD-specific taxa were increased in CD/UC (13 up- and 2 down-regulated ASVs), potentially implying a more direct causal association to pathogenesis and gut inflammation.

According to some embodiments, 13 CD/UC-specific upregulated ASVs showed enrichment for salivary bacteria.

According to some embodiments, the non-specific dysbiosis index (NSDI) utilizes the shared disease-associated ASVs.

According to some embodiments, the NSDI successfully differentiates between most cases and controls across a wide variety of diseases.

According to some embodiments, the basic unit of independent measurement is the disease cohort rather than samples.

According to some embodiments, increase in Enterobacteriaceae taxa and a decrease in the butyrate producing Faecalibacterium prenzutzi were consistent with alterations seen across multiple diseases and are likely not IBD-specific.

Advantageously, the non-specific signal includes increased rather than just decreased bacterial ASVs.

According to some embodiments, limiting the analyses to similar sequencing region of the 16S (V4) narrow the number of available studies, but advantageously enable an ASV, rather than taxonomy-based, comparison.

According to some embodiments, the large number of ASVs that show similar decrease in relative abundance in a large variety of diseases may imply that these bacteria are affected by a general response of the host to disease, and can potentially reflect a general change in immune and/or metabolic states.

Advantageously, ASV sequence level, rather than taxonomy level, is leading to an enhanced phylogenetic resolution compared to taxonomy-based comparison, which is usually limited to the genus level for 16S amplicon sequencing.

According to some embodiments, the non-specific health-associated taxa included Faecalibacterium prausnitzii (NCBI NZ_CP030777) and Lachnospiraceae Coprococcus.

According to some embodiments, the non-specific disease-associated taxa include Clostridium XIVa (also known as Enterocloster, NCBI NZ_CP022464), Lactobacillus salivarius (NCBI NZ_CP011403), and Micrococcaceae Rothia (NCBI NZ_ACV001000020).

According to some embodiments, cellular community pathways were more common in disease-associated ASVs, and those were more specifically associated with bacteria manifesting strong disease associated quorum sensing. According to some embodiments, the NSDI comprises less quorum sensing ASVs than disease-associated ASVs. According to some embodiments, the NSDI comprises less than 30%, less than 20%, less than 10%, less than 5% of the quorum sensing ASVs.

According to some embodiments, the performance classifiers, such as but not limited to a random forest classifier, on all pairs of disease cohorts was exhaustively tested by training on one disease and predicting the second disease-cohort.

According to some embodiments, a classifier trained on a single IBD disease cohort also classified, with relatively good performance, disease/control states in lupus, schizophrenia, or Parkinson's from different cohorts.

According to some embodiments, classifiers may differentiate between different diseases, when trained on datasets containing multiple diseases.

In accordance it was found to be recommended and advantageous for disease state prediction, to train the microbiome-based classifier also on a large set of diseases, in order to prevent misdiagnosis that can result from a classifier trained on a single disease cohort.

According to some embodiments, the presence of disease non-specific bacterial response enables estimation of the general host dysbiosis state.

According to some embodiments, using the set of disease non-specific ASVs as a marker of dysbiosis provides better performance in assessing the patient state of various diseases.

Advantageously, NSDI can successfully differentiate between most cases and controls across a wide variety of diseases.

According to some embodiments, a general dysbiosis index can serve as an additional tool for prioritizing fecal microbiota transplant (FMT) donors, where it is desired to obtain samples from donors with lower probability of suffering from a disease.

Advantageously, the ability to identify specific microbial disease patterns can help direct toward ASVs bacteria taking part more directly in the actual pathogenesis, aid in specific diagnostics, and support the design of potential future interventions.

According to some embodiments, ASVs that are specifically associated with CD/UC were defined.

According to some embodiments, utilizing the large number of IBD disease cohorts in the present disclosure, 15 UC/CD-specific bacterial ASVs were identified, out of which 13 show a higher increase in IBD compared to other disease (13 ASVs having sequences set forth in any one of SEQ ID NO: 131-143), and 2 show a decrease (2 ASVs having sequences set forth in any one of SEQ ID NO: 129-130).

According to some embodiments, the ASVs included in the IBD-specific signal include at least 2, at least 3, at least 5, at least 8, at least 10, at least 12, or all of the ASVs listed in SEQ ID NO: 129-143. Each possibility is a separate embodiment.

According to some embodiments, the ASVs included in the IBD-specific signal include at least 2, at least 3, at least 5, at least 8, at least 10, at least 12, or all of the ASVs listed in SEQ ID NO: 129-141. Each possibility is a separate embodiment.

According to some embodiments, the ASVs included in the IBD-specific signal include at least 1, or all of the ASVs listed in SEQ ID NO: 142-143 Each possibility is a separate embodiment.

According to some embodiments, the ASVs listed in SEQ ID NO: 142-143 are also included in the non-specific signal, but they are more strongly up-regulated in IBD-diseases than in the other diseases. According to some embodiments, the ASVs listed in SEQ ID NO: 142-143 are up-regulated at least 1.2 times, at least 1.5 times, at least 2 times, or at least 3 times more strongly as part of the IBD specific signal than as part of the non-specific signal.

According to some embodiments, ASVs are up-regulated at least 1.1 times, at least 1.2 times, at least 1.5 times, at least 2 times, at least 3 times, or at least 5 times more strongly in cases compared to controls.

According to some embodiments, ASVs are down-regulated at least 1.1 times, at least 1.2 times, at least 1.5 times, at least 2 times, at least 3 times, or at least 5 times more strongly in cases compared to controls.

According to some embodiments, bacteria having the potential to be more specifically associated with the IBD pathology include taxa from the Gemellaceae family (NCBI NZ_CP046314), Veillonellaceae Dialister (NCBI NZ_QWKU01000001), Lachnospiraceae Blautia producta (NCBI NZ_CP030280), and Streptococcaceae Streptococcus (NCBI NZ_FMIX01000001 and NZ_JYGP01000001).

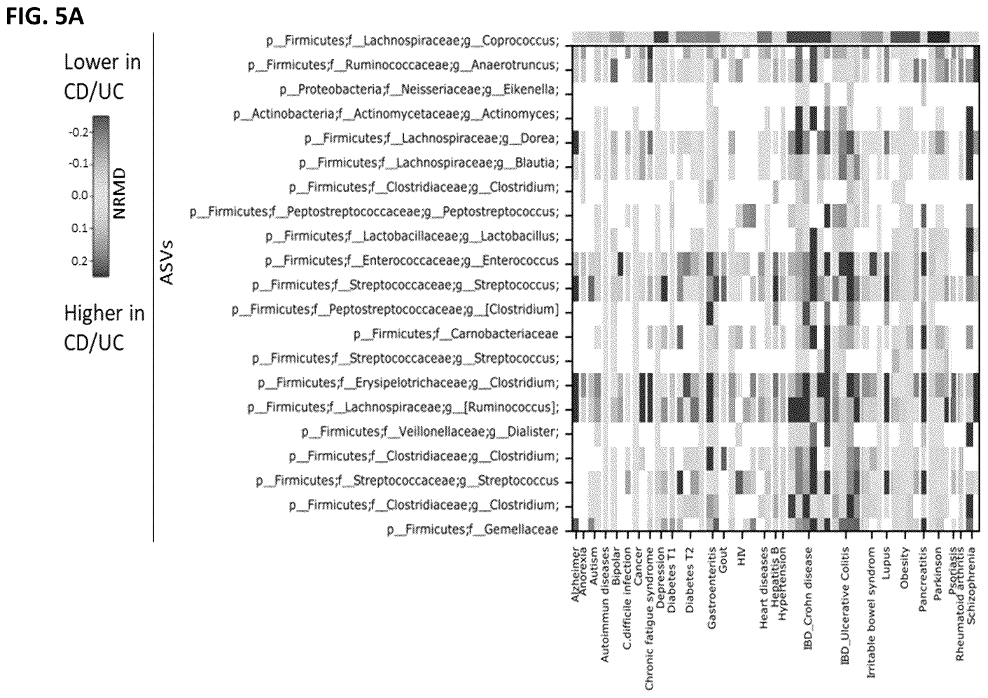

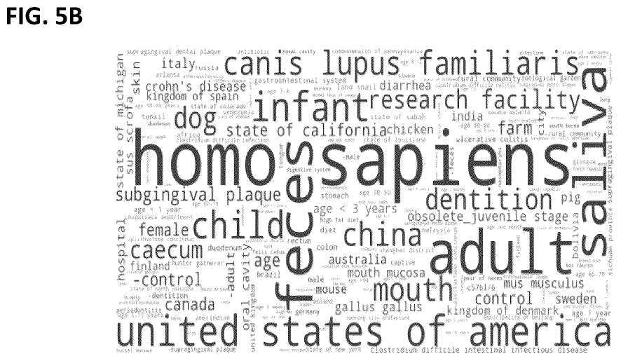

According to some embodiments, the UC/CD specific ASVs were also part of a signal that was related to the non-specific signals but those showed significant increase in CD and UC (reference is made to the heatmap in FIG. 5A).

According to some embodiments, the UC/CD specific ASVs that were also part of a signal that was related to the non-specific signals but showed a significant increase in CD and UC include those ASV having their sequence listed in SEQ ID NO: 142-143.

As used herein, in some embodiments, the UC/CD specific ASVs that were also part of a signal that was related to the non-specific signals, showed a significant increase of at least 2 fold, at least 3 fold, at least 5 fold, in CD and UC relative other diseases.

According to some embodiments, UC/CD specific bacteria were Clostridium perfringens (NCBI NC 008261), Ruminococcus gnavus (NCBI NZ_CP027002), Enterococcaceae Enterococcus (NCBI NZ_KB946332), and Lachnospiraceae Dorea (also known as Faecalimonas, NCBI NZ_BHEO01000008).

According to some embodiments, many of these UC/CD specific bacteria have been shown to be present in saliva samples.

According to some embodiments, herein disclosed is a robust non-specific general response dominated by reduction of microbial ASVs with a smaller group of bacteria that were up-regulated in a large array of diseases.

According to some embodiments, these non-specific changes reflect a general response of the body to pathologic condition linked with altered immune and/or metabolic sick state.

According to some embodiments, the defined increased IBD-specific taxa imply to a more direct causal association to pathogenesis and gut inflammation.

According to some embodiments, the non-specific dysbiosis index (NSDI) utilizing the shared disease-associated ASVs can successfully differentiate between cases and controls across a plurality of diseases.

According to some embodiments, the non-specific dysbiosis index (NSDI) utilizing the shared disease-associated ASVs can be used as an additional tool for prioritizing samples and donors with lower disease probability.

Advantageously, 128 ASVs have similar behavior across multiple disease, and therefore associated with general disease state. According to some embodiments, most (97 ASVs) showed reduced abundance shared across different diseases, and only 31 showed higher abundance across different diseases.

According to some embodiments, the relative taxonomy composition of the two groups shows Bacteroidetes comprise 11 of the 97 nonspecific health-associated ASVs, whereas none were within the 31 disease-associated ASVs.

According to some embodiments, Actinobacteria taxa were more abundant within disease-associated ASVs (3/31) compared to health-associated ASVs (1/97). According to some embodiments, Lactobacillales order (Firmicutes phyla) were significantly enriched within the disease-associated ASVs (5/31 vs. 0/97 for disease-and health-associated ASVs respectively.

According to some embodiments, the terms associated with those 31 disease-associated ASVs using dbBact (http://dbbact.org/), advantageously indicated that those tended to be associated with in feces and adult humans, and lower in control and rural communities.

According to some embodiments, KEGG ontologies which are significantly more common in the health or disease-associated ASVs included 23 pathways that were identified, 9 that were more common in disease and 14 more common in controls. According to some embodiments, pathways more common in disease included carbohydrate metabolism and cellular community pathways specifically associated with quorum sensing genes, whereas pathways more common in controls included metabolism of cofactors and vitamins, and amino acid metabolism.

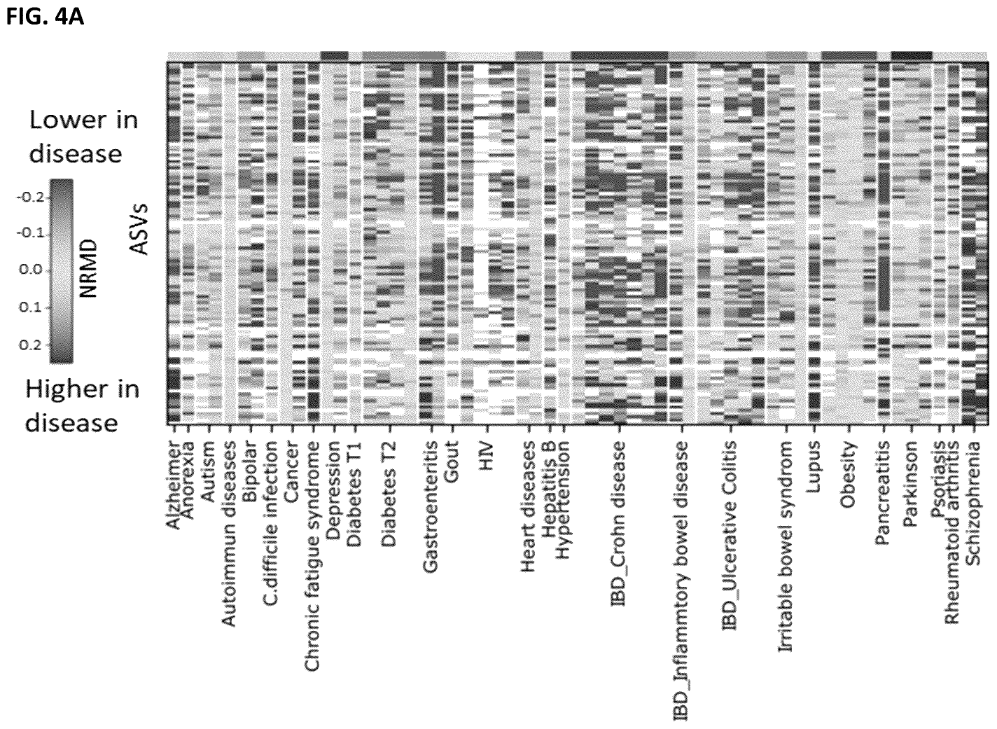

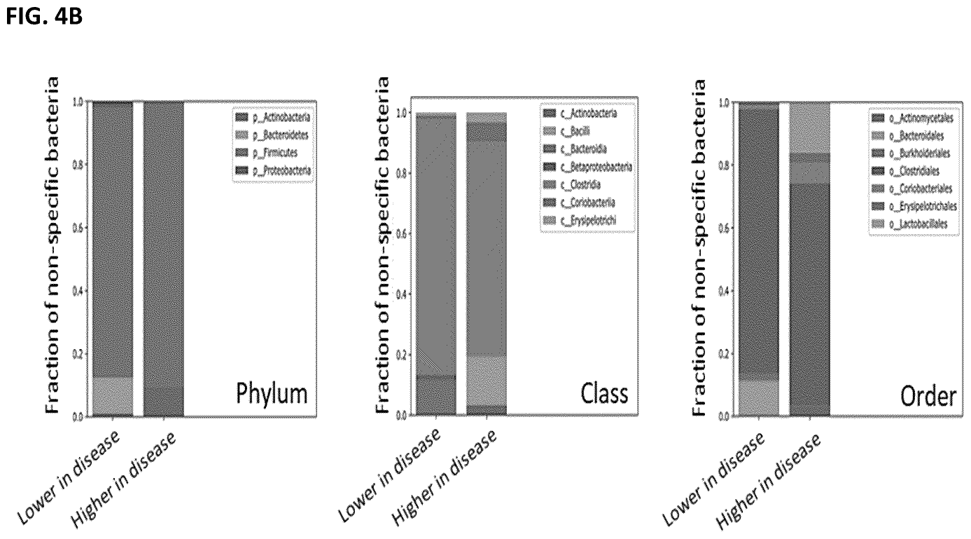

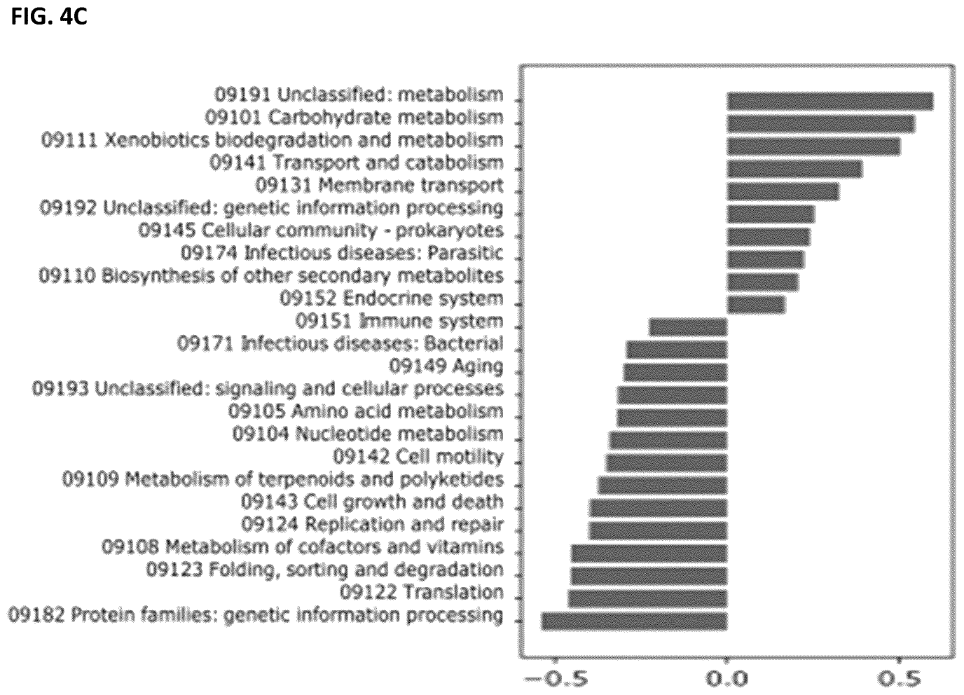

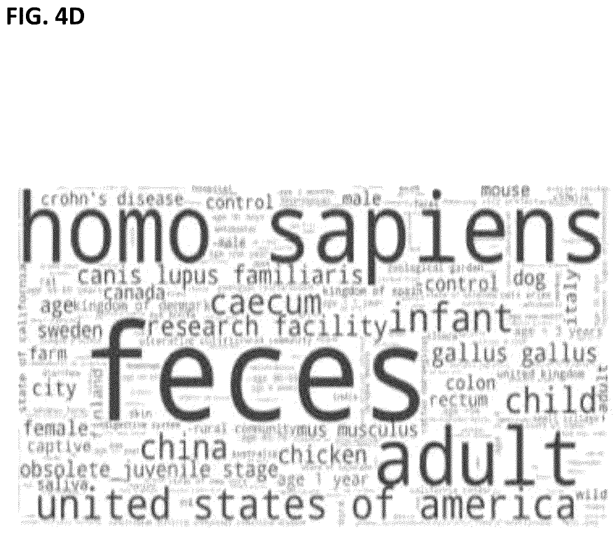

Reference is now made to FIG. 4 showing analyses of non-specific microbial signal shared across diseases.

Advantageously, 15 ASVs were significantly related to UC and CD, with 13 showing a higher NRMD between UC and CD cases and controls, and 2 showing a decrease NRMD between UC/CD and controls.

According to some embodiments, those specific CD and UC enriched ASVs included taxa form Gemellaceae, Veillonellaceae, Neisseriaceae and Streptococcaceae families.

According to some embodiments, term enrichments of those 13 CD/UC-associated specific ASVs using dbbact database, showed enrichment for microbial taxa seen in saliva and taxa lower in controls.

Reference is now made to FIG. 5 showing analyses of CD/UC “specific” ASVs.

Advantageously, according to some embodiments, the classifier performs well in identifying many sick vs. healthy state based on the non-specific shared microbial disease signal due to the ability of the classifier to separate between disease cases and controls across cohorts and diseases, indicating that the disease-associated signal is surprisingly strong and consistent and can be traced despite studies-specific processing or geography.

Advantageously, according to some embodiments, the NSDI recapitulates a consistent change that persists across multiple diseases, therefore performing better than the previously published CD dysbiosis index, indicating that NSDI can successfully differentiate between most cases and controls across a wide variety of diseases

According to some embodiments, the herein disclosed non-specific dysbiosis index (NSDI) and machine learning based classifier exemplify the advantage of the herein disclosed process/method for computing a dysbiosis index score (“DI score”), that relies on large datasets which recapitulate a consistent change that persists across multiple diseases, in order to differentiate between healthy controls and disease cases across a plurality of diseases, thereby assessing/classifying the health status associated microbiome profile based on the DI score.

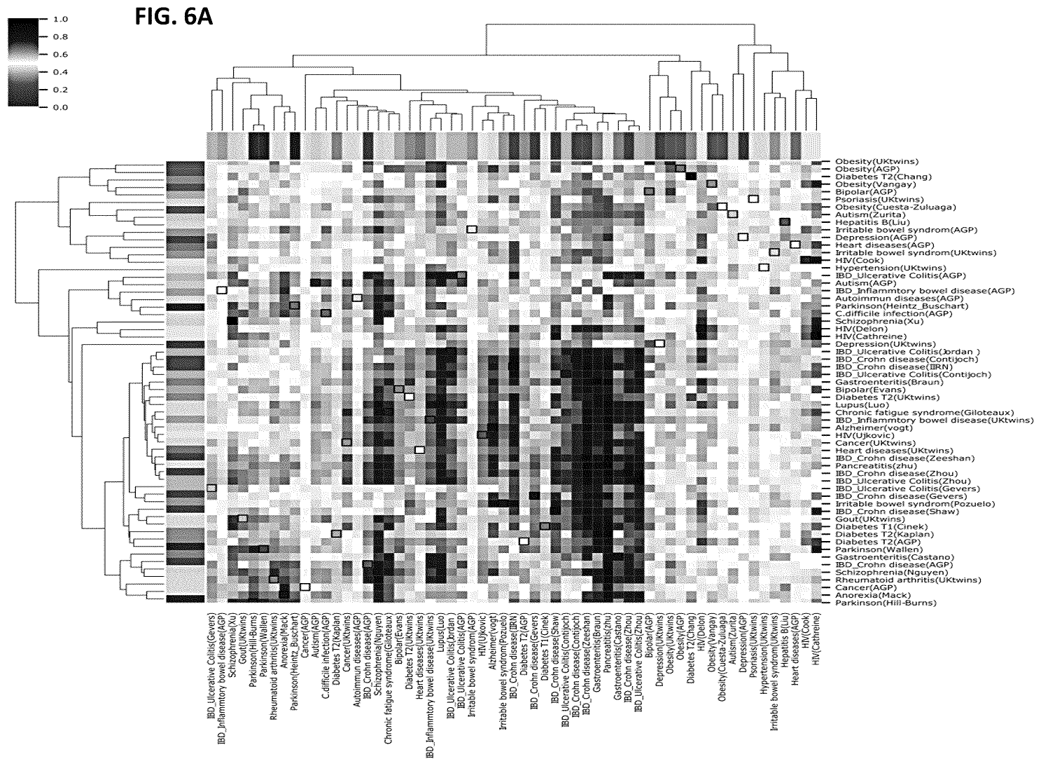

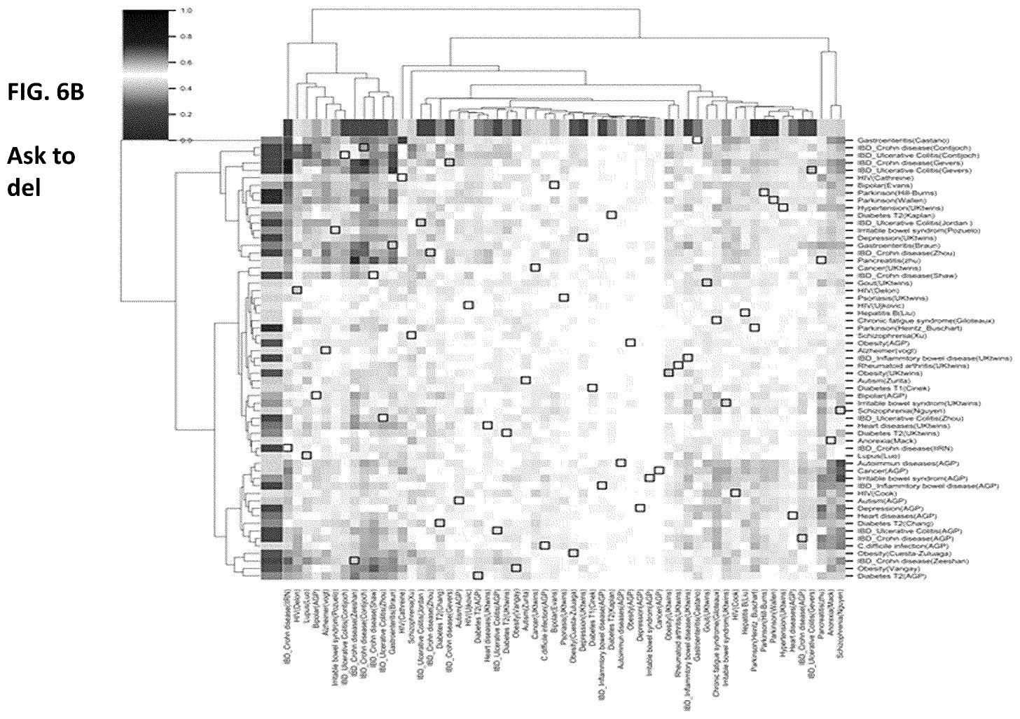

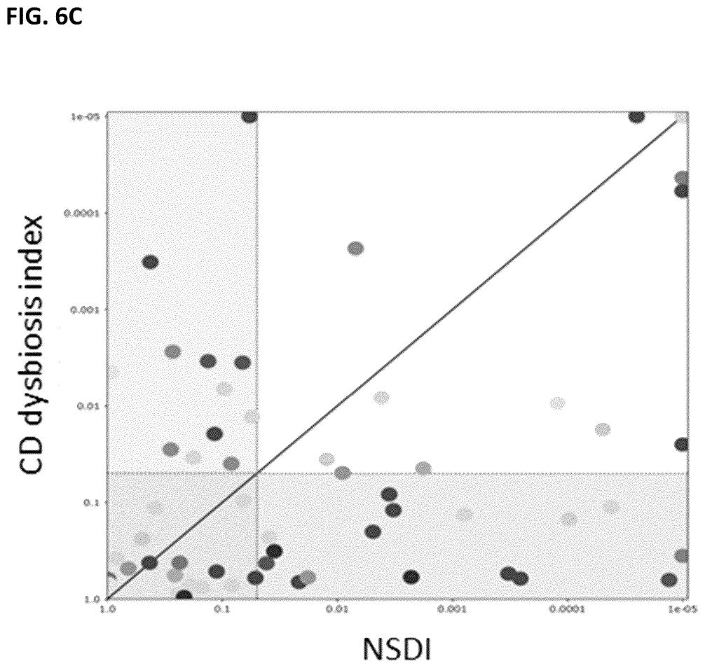

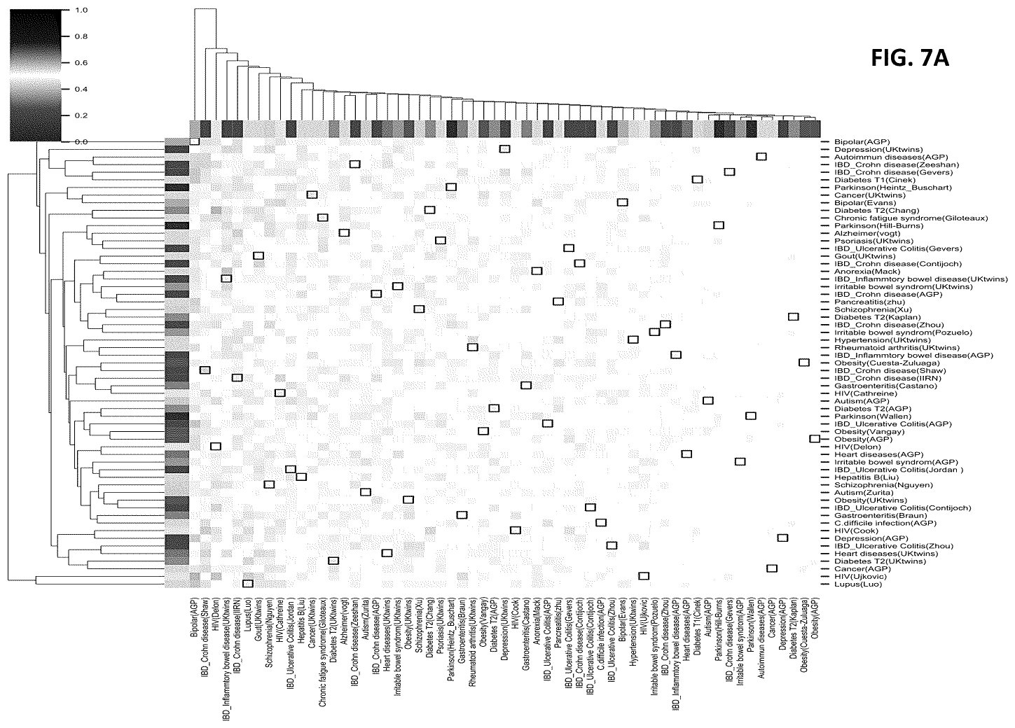

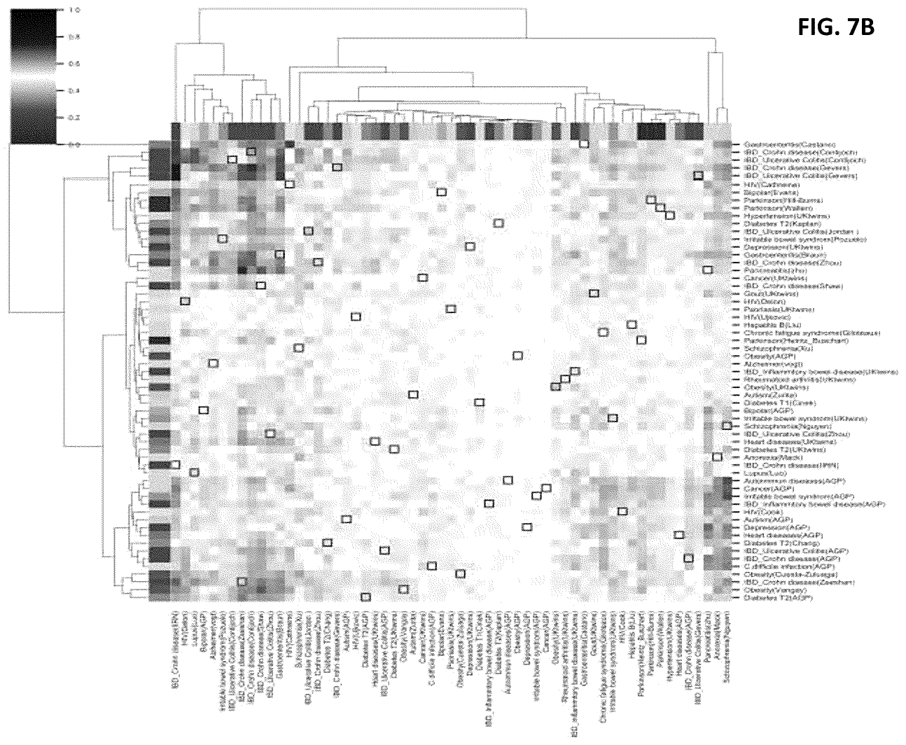

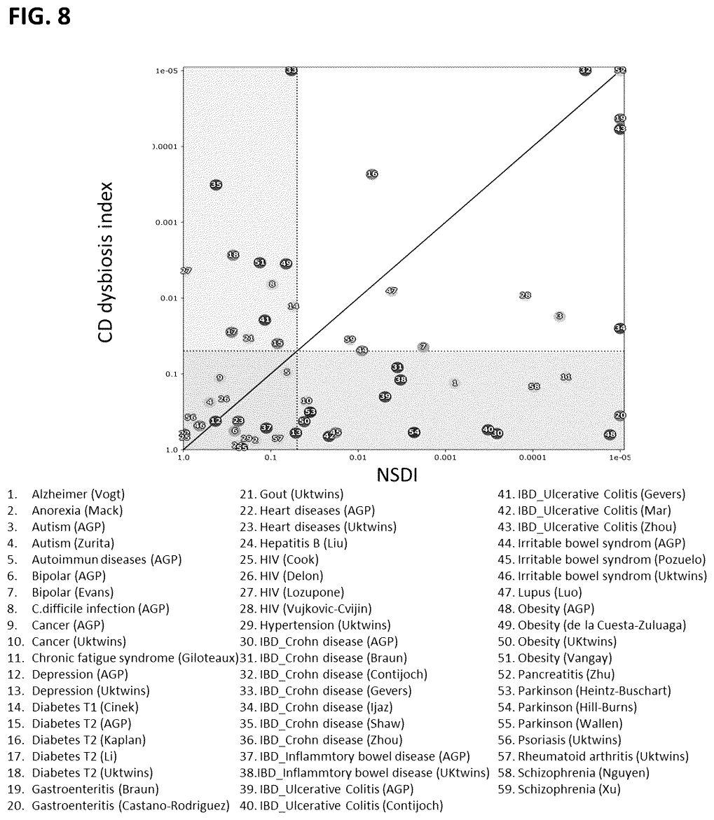

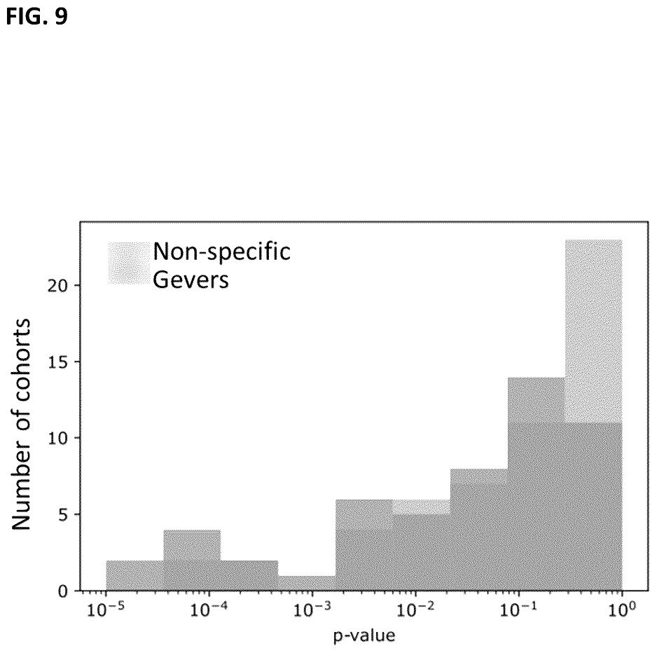

Reference is now made to FIG. 6, FIG. 7, FIG. 8, and FIG. 9 presenting the dysbiosis index and disease classifier.

According to some embodiments, NSDI shows ability to significantly differentiate between cases and controls in diseases other than those used to generate the NSDI.

According to some embodiments, NSDI significantly differentiates between cases and controls of samples obtained from lung cancer patients.

According to some embodiments, NSDI significantly differentiates between cases and controls of samples obtained from subjects hospitalized in rehab.

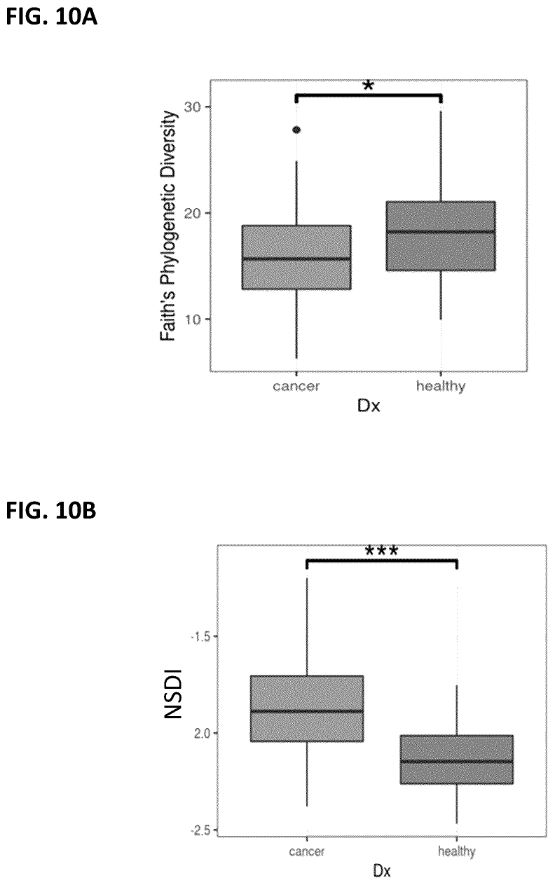

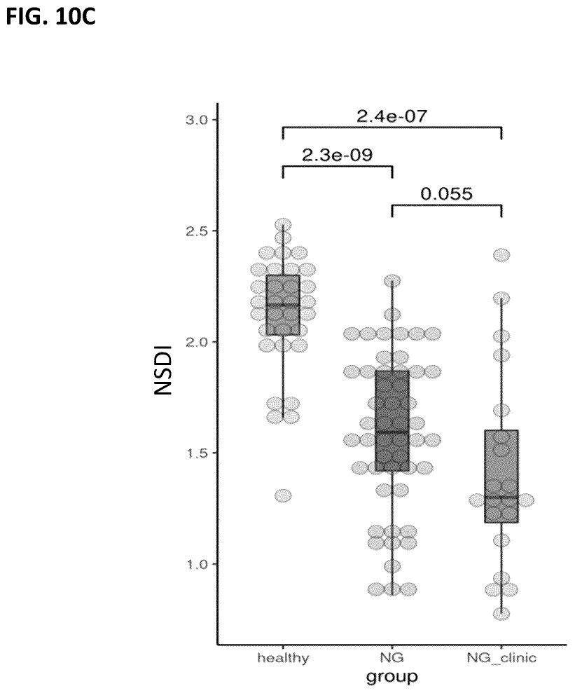

Reference is now made to FIG. 10

The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

### Methods

Study search and disease-cohort selection strategy—A search for case-control (disease-cohort) 16S amplicon sequencing studies was established using specific keywords in Google Scholar and dbBact (http://dbbact.org/), and by following references in meta-analyses and related case-control studies. Only studies with at least 20 subjects, with stool or biopsies samples that were sequenced using hypervariable V4 region, and for which the Fastaq files were publicly available for download or obtained after a specific request, were included. List of diseases with case-control comparison from those studies are summarized in Table 1.

TABLE 1
Description of case-control comparison included in the present
disclosure.

| Disease (n = no. of studies) | Cases | Controls | Country |
|---|---|---|---|
| Anorexia (n = 1) | 55 | 54 | Germany |
| Autism (n = 2) | 135 | 179 | USA |
| | 27 | 31 | Ecuador |
| Autoimmune diseases (n = 1) | 298 | 522 | USA |
| Alzheimer (n = 1) | 25 | 25 | USA |
| Bipolar (n = 2) | 6 | 11 | USA |
| | 115 | 64 | USA |
| Cancer (n = 2) | 22 | 23 | United Kingdom |
| | 220 | 405 | USA |
| C. difficile infection (n = 1) | 32 | 61 | USA |
| Chronic fatigue syndrome (n = 1) | 49 | 39 | USA |
| Depression (n = 2) | 272 | 388 | USA |
| | 75 | 40 | United Kingdom |
| Diabetes T1 (n = 1) | 71 | 103 | Multi |
| Diabetes T2 (n = 4) | 36 | 64 | USA |
| | 349 | 275 | Multi |
| | 20 | 21 | United Kingdom |
| | 19 | 39 | China |
| Gastroenteritis (n = 2) | 203 | 31 | Israel |
| | 75 | 423 | Australia |
| Gout (n = 1) | 17 | 16 | United Kingdom |
| Heart diseases (n = 2) | 106 | 168 | USA |
| | 32 | 31 | United Kingdom |
| Hepatitis B (n = 1) | 35 | 33 | China |
| HIV (n = 4) | 41 | 79 | USA |
| | 18 | 14 | USA |
| | 22 | 12 | USA |
| | 82 | 84 | Netherlands |
| Hypertension (n = 1) | 124 | 74 | United Kingdom |
| IBD (n = 2) | 107 | 194 | USA |
| | 13 | 17 | United Kingdom |
| IBD-Crohn's Disease (n = 7) | 31 | 58 | USA |
| | 37 | 29 | Israel |
| | 144 | 35 | multi |
| | 479 | 116 | USA |
| | 23 | 70 | United Kingdom |
| | 15 | 10 | USA |
| | 183 | 68 | China |
| IBD-Ulcerative Colitis (n = 5) | 39 | 66 | USA |
| | 109 | 35 | multi |
| | 85 | 29 | USA |
| | 30 | 13 | USA |
| | 73 | 69 | China |
| Irritable bowel syndrome (n = 3) | 444 | 717 | USA |
| | 110 | 66 | Spain |
| | 21 | 22 | United Kingdom |
| Lupus (n = 1) | 14 | 17 | USA |
| Obesity (n = 4) | 503 | 436 | USA |
| | 172 | 269 | Colombia |
| | 24 | 151 | multi |
| | 33 | 40 | United Kingdom |
| Pancreatitis (n = 1) | 145 | 35 | China |
| Parkinson's (n = 3) | 46 | 37 | Germany |
| | 213 | 135 | USA |
| | 524 | 316 | USA |
| Psoriasis (n = 1) | 43 | 42 | United Kingdom |
| Rheumatoid arthritis (n = 1) | 34 | 40 | United Kingdom |
| Schizophrenia (n = 2) | 18 | 20 | USA |
| | 44 | 40 | China |
| Total 28 disease | 6337 | 6501 | |

Only one sample per patient was kept in cases where several samples were obtained. In studies that had UC/CD patients, each disease was considered as a separate disease-cohort, and the controls were randomly divided between the two case-control comparisons. In this meta-analysis, two large cohorts with multiple diseases were included: the UK Twins cohort (https://twinsuk.ac.uk/), and the American Gut Project (AGP) cohort (http://humanfoodproject.com/americangut/). In cases where controls were not defined (such as in the AGP and UK twins), controls were considered as those having BMI<30 and not taking oral medications other than supplements and vitamins. Controls were then randomly divided (taking into account age, gender, and Country of origin when applicable) between the different disease-cohorts in each cohort, so that each control sample participated only in one disease-cohort. To define obesity, subjects with BMI 30 or greater were included in the obesity group. Only subjects with single disease category were included and dropped those with two or more diseases, but retained IBD patients who had also IBS or autoimmune disease as those may co-exist, which were then included in the analysis as IBD patients.

V4 16S raw data processing—Single-end reads were left trimmed to begin at the end of the 515′F primer, and right trimmed to a total length of 150 bp. Reads from each cohort were then aligned and denoised using the Deblur pipeline in qiime2 using default parameters, resulting in a list of per-study bacterial Amplicon Sequence Variants (ASVs). Those bacteria were filtered across all studies included in this analysis in order to prevent sample-storage associated bacteria. ASV taxonomic classification was performed using a naive Bayes fitted classifier, as essentially known in the art, trained on the August 2013 99% identity Greengenes database, for 150 bp long reads and the corresponding primers set as implemented in qiime2 command: qiime2 feature-classifier classify sklearn with default parameters.

Measuring study and disease effect sizes using standard approach and using aggregated meta-sample approach—In order to mitigate the effect of different cohort sizes in different studies, up to 23 samples were randomly chosen from each case/control disease-cohort, resulting in 2375 samples. Then, a standard (sample-based) beta diversity analysis and the aggregated meta-sample approach were used.

For the aggregated approach—each disease and control group of up to 23 samples was then aggregated to a single meta-sample using the mean frequency of each ASV. Bray-curtis distances between meta-samples were then calculated within and between studies, and PCoAs were generated. Significance for the difference in distances between cohorts and disease state were calculated using the non-parametric Mann-Whitney test. PCoAs are specifically shown for distances calculated between disease and controls within each disease cohort.

For the standard approach—Bray-Curtis distances, followed by PERMANOVA test were applied to quantify the contribution of different factors affecting the gut microbial composition. Analysis was performed on all 2375 samples, or only on the mean aggregated single meta-sample in the control and disease groups in each of the 59 disease-cohort together (59 disease states and 59 controls), only on the 59 controls, and only on 59 diseases. The quantifications of variance were calculated using PERMANOVA with the adonis function in the R package Vegan (vegan: Community Ecology Package. R package version 2.5-6.https://CRAN.R-project.org/package=vegan), on the Bray-Curtis distance values, of the actual relative abundance ASV or the mean aggregated single meta-sample ASV per cases and controls group as indicated. The total variance explained by each variable was calculated independently of other variables (that is, as the sole variable in the model).

Calculation of the per-ASV effect size within each disease-cohort, followed by statistical analyses to define specific and non-specific disease response—Selection of ASVs and calculation of the per-ASV effect size: In order to create the list of per disease-cohort ASV case/control effect, only ASVs that show significant differential abundance (between cases and controls) were used within at least one disease-cohort. These ASVs were identified independently within each disease-cohort using a non-parametric rank mean test with dsFDR multiple hypothesis correction (FDR<0.25), based on a random subset of 23 cases and 10 controls samples per disease-cohort, in order to avoid the dominance of disease-cohorts with a large number of samples. A unified list of ASVs showing differential abundance in at least one study was then generated. For each of those ASVs, the direction of change and the effect size (normalized rank mean difference between the mean of cases and controls) were calculated in each case-control comparison using not the 23/10 sample subset but rather all samples in the disease-cohort across all studies. The normalized rank-mean difference (NRMD) is scaled to be in the range of −1 to 1 (independent of the number of samples in each group) and was calculated using the formula:

$$\\ N R M D = ( mean ( {G}_{case} ) - mean ( {G}_{control} ) ) / ( \frac{n ( {G}_{case} ) + n ( {G}_{control} )}{2} )$$

Where NRMD is the normalized effect size, Gcase/Gcontrol represents the ranked frequencies of the case/control for the given ASV, and n(G) represents the number of samples in group G.

Ratio based beta diversity analysis: To see the similarity/dissimilarity between the different studies, the ratio between cases and controls in each cohort were used for cluster analysis by calculating the Bray-Curtis distances on the ratios. Comparisons were performed between each two cohorts only on ASVs found in both. For each disease-cohort, the log-ratio of the mean abundance between healthy and disease samples was calculated for each ASV, resulting in a single vector (of length N, where N is the number of unique ASVs observed in the disease-cohort). This vector was used as a “sample” for disease cohort beta-diversity. For distance calculation between two such disease cohort “samples”, we used a modified Bray-Curtis distance metric as follows:

$$\\ D ( {S}_{i} , {S}_{j} ) = {{\sum }_{x}}_{{S}_{i , x} \neq 0 and {S}_{j , x} \neq 0} \frac{\vert {S}_{i , x} - {S}_{j , x} \vert}{\vert {S}_{i , x} \vert + \vert {S}_{j , x} \vert}$$

Where Si,x denotes the sick/healthy log-ratio of ASV x in sample Si

This modified Bray-Curtis distance is similar to the classic Bray-Curtis distance calculated only on ASVs present in both samples. This modified Bray-Curtis metric was opted to reduce the effect of the different disease-cohorts (i.e., different populations/extraction protocols etc.) that can lead to the disappearance of some ASVs in specific cohorts, therefore prohibiting the determination of the sick/healthy ratio for the ASV in the specific cohort.

A distance matrix for all disease-cohort pairs was calculated using this modified Bray-Curtis metric. This matrix was then used for the generation of a PCoA depicting disease-cohort similarity using qiime2.

Identification of shared (“non-specific”) ASVs: The direction of the effect size (i.e. higher (lower) NRMD in cases or controls for the given disease) of ASVs not associated with a given disease should follow a 0.5/0.5 binomial distribution. Therefore, in order to identify shared (“non-specific”) ASVs, ASVs whose effect size direction significantly differs from 0.5/0.5 binomial were searched for (i.e. ASVs that are higher (lower) in disease compared to controls in a significant number of different diseases). This was implemented by using a binomial test (p=0.5) on the sign of the NRMD between case and control (only on studies where the ASV was present), followed by Benjamini-Hochberg FDR control (FDR<0.1). To prevent bias by disease with many disease-cohorts, different disease-cohorts with the same disease were aggregated to a single entry (prior to the binomial test) with the NRMD defined as the mean of the NRMD of all cohorts with the same disease. The analysis was performed only on ASVs present in at least 4 disease-cohorts.

Identification of IBD specific ASVs: IBD specific ASVs were defined as ASVs showing significantly higher (or lower) NRMD values in CD and UC fecal disease-cohorts in comparison to other disease-cohorts. These IBD specific ASVs were identified using a rank-mean test (implemented in Calour) on the NRMD in all studies (i.e., 10 CD and UC vs. 45 with other disease, not including the 2 non-specific IBD diagnosis disease cohorts, and the 2 diseases cohorts that used biopsies rather than fecal samples), with dsFDR correction (FDR=0.1).

dbBact terms word clouds: ASVs either specific and non-specific, that were shown to be related to disease, were compared to all the annotations in the dbbact database to search for words related to those ASVs (i.e., diseases, geographical locations, bacterial main niches in the body, and habitant for those bacteria).

Functional enrichment analysis in non-specific bacteria—Functional analysis was performed using per-ASV prediction of KEGG ontologies obtained using PICRUSt2, as essentially known in the art. We then searched for KEGG ontologies present significantly higher in non-specific bacteria increasing (decreasing) in disease compared to controls (e.g., a KEGG ontology more common in bacteria that increase (decrease) in multiple diseases compared to the bacteria that decrease (increase) in controls) using rank-mean test (implemented in Calour) with dsFDR multiple hypothesis correction (FDR<0.1).

Classifier building and performance—For the classification, each cohort was randomly subsampled to a maximum of 23 healthy and 23 disease samples. For each pair of cohorts (train, predict), ASVs were filtered, keeping only ASVs present in both cohorts. A random forest classifier (implemented in scikit-learn version 0.23.1, using default parameters, 100 trees per forest) was trained on the disease/control samples in the training cohort. The trained classifier was then used to predict the disease/control status of the prediction cohort, and false and true positive rates and AUC were calculated using scikit-learn. In the special cases where the train and predict cohort were the same (i.e. assessing the classifier predictions on the same cohort), the cohort samples were randomly split to 2/3 of the samples to be used as the training cohort, and the remaining 1/3 of the samples used as the prediction cohort.

Three random null-models were used: In the random-prediction model, labels of the prediction cohort (i.e., disease/control) were randomly permuted prior to the classifier prediction. In the random-training model, labels of the training cohort (again disease/control) were randomly permuted prior to the training (thus leaving intact the disease/control differences in the prediction cohort). In the third model, labels of the prediction cohort were randomly permuted prior to the classifier prediction, and the labels of the training cohort were randomly permuted prior to the training.

Dysbiosis index—For a given sample (containing ri reads for ASV 1 . . . n):

S=(r₁,r₂, . . . ,rₙ),

\tilde{S}=rank(S)

per-sample reads were first rank-transformed, and the dysbiosis index is then defined as the normalized log ratio of the sum of the ranks of the up and down regulated ASVs:

$$\\ D N {S}_{S} = {log}_{2} ( \frac{{\sum }_{i ∈ up \_ nonspecific} \tilde{{s}_{ι}}}{{\sum }_{i ∈ down \_ nonspecific} \tilde{{s}_{ι}}} \cdot \frac{\vert down\_nonspecific \vert}{\vert up\_nonspecific \vert} )$$

Where, up_non-specific, down_non-specific, represent the group of disease non-specific ASVs higher and lower in disease respectively.

The taxonomy based dysbiosis index was calculated using the below recited formula, implemented for the denoised data using the taxonomy assigned to each ASV:

$$\\ D T A {X}_{S} = {log}_{2} ( \frac{{\sum }_{i ∈ up \_ taxonomies} {s}_{i}}{{\sum }_{i ∈ down \_ taxonomies} {s}_{i}} )$$

Where up_taxonomies, down_taxonomies are the lists of taxonomies higher and lower in Crohn's disease.

### Results

### Example 1: Uniform Case-Control Analytic Pipeline Starting From V4 Amplicon Raw Data

A search was established to identify case-control disease-cohorts that used V4 16S rRNA amplicon sequencing. This resulted in 34 different original studies, 59 disease-cohorts spanning 28 diseases and 12,838 subjects (Table 1). Two large cohorts with multiple diseases were included: the UK Twins (https://twinsuk.ac.uk/), and the American Gut Project (AGP) cohorts (http://humanfoodproject.com/americangut/). In those and in studies where several diseases were investigated, controls were randomly divided between the different disease-cohorts in each original study. Only one sample per patient was included in cases where several samples per person were obtained. Notably, cohorts originating from different geographical regions including North America, Europe, Middle East, and Asia were included, as indicated in Table 1.

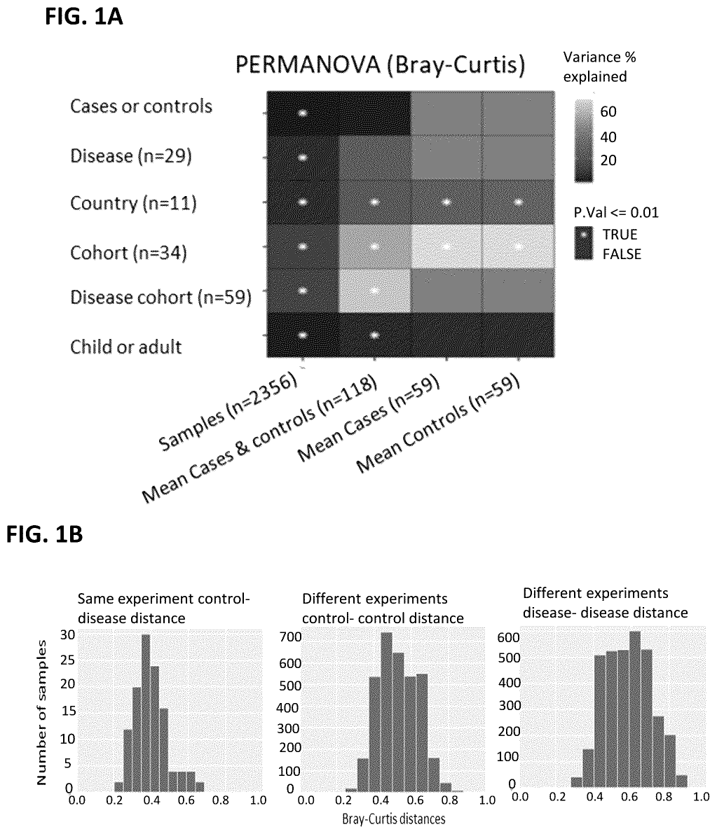

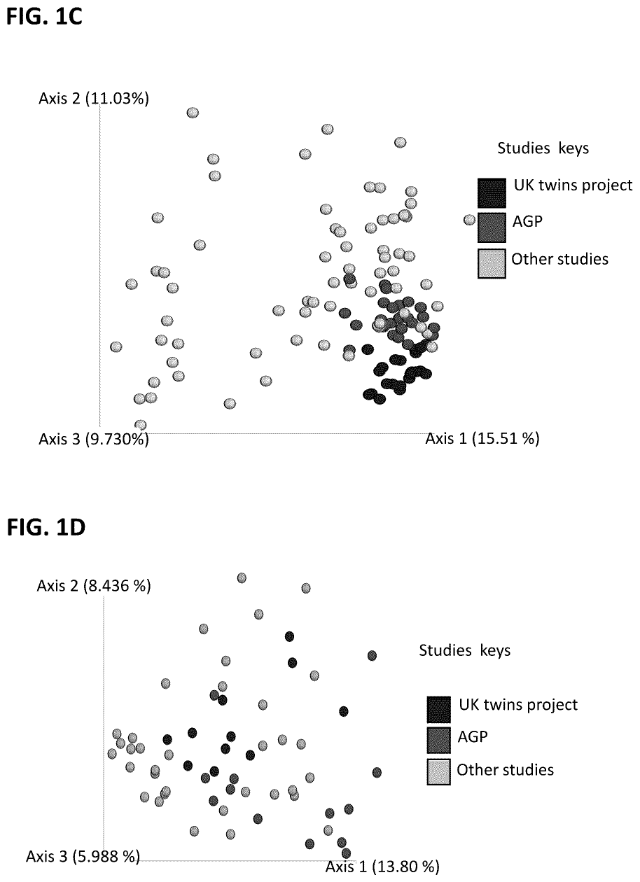

For each original study, raw reads from all samples were trimmed and denoised using Deblur, resulting in per-sample relative abundance of bacterial amplicon sequence variants (ASVs). Since all cohorts used the same V4 16S rRNA region, it was possible to process, combine, and analyze all cohorts together at the ASV sequence level, rather than taxonomy level, thus leading to an enhanced phylogenetic resolution compared to taxonomy-based comparison, which is usually limited to the genus level for 16S amplicon sequencing. Since the data originated from multiple cohorts, first the contribution of the sample source to the microbial variability was examined. Study-specific variation was the major confounder in microbial data analyses across different studies (explained variance using PERMANOVA test on Bray-Curtis distances FIG. 1A), likely due to sample collection and processing methods, as well as differences in the populations studied. This was further supported by measuring distances (beta-diversity) between sample-groups within and between studies (FIG. 1B), showing significantly higher distances between controls from different studies in comparison to control and disease samples from the same study; mean Bray-Curtis distances of 0.4, 0.53, 0.58 for within study case-control, between-study only controls and between-study only cases (non-parametric Mann-Whitney p-value<1E-10 for all group comparisons).

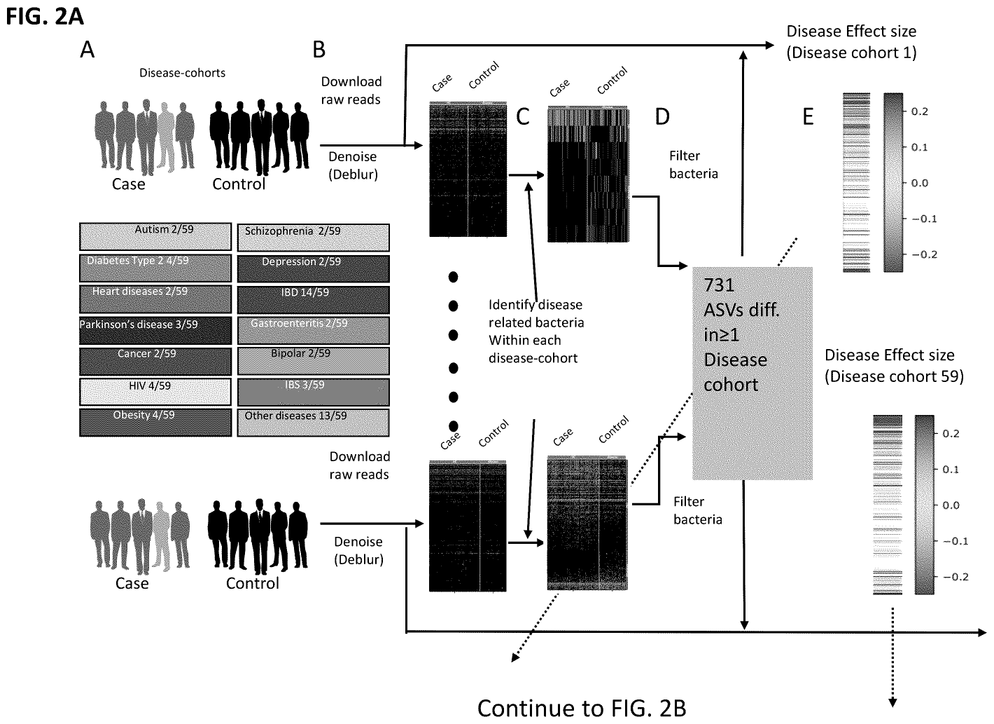

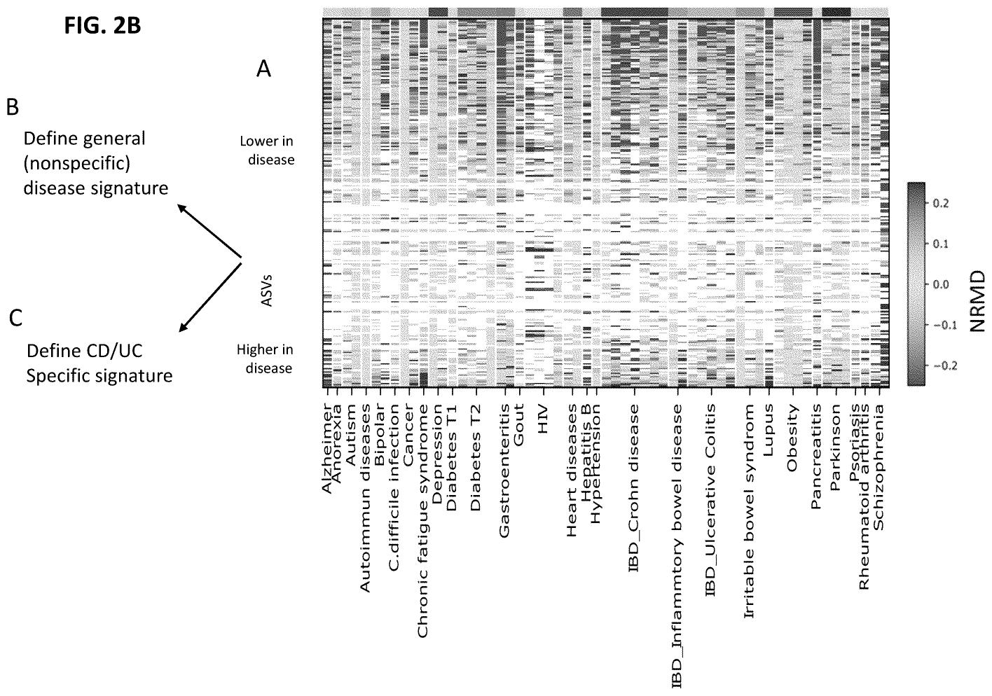

To overcome these study-specific signals and facilitate comparison of signals across the different cohorts, a per-study effect size-based pipeline was devised (summarized in FIG. 2A). The concept is to calculate the disease-dependent effect-size for each ASV within each disease-cohort separately, and then perform the metanalysis on these effect-sizes rather than on the ASV base frequencies, which are strongly affected by the studies. Specifically, ASVs showing a potential case/control difference in at least one study were identified first. Since this initial screening is used to identify ASVs that are then further selected using additional statistical tests, a relatively small number of samples per study with a high FDR was opted for, in order to include as many studies and differential abundance ASVs as possible. This was done by independently running a differential abundance test (non-parametric rank mean test with dsFDR<0.25 multiple hypothesis correction) on a random subset of 23 cases and 10 controls from the disease-cohort (to avoid bias due to different cohort sizes). These ASVs from all the disease cohorts were then combined to a unified list of 731 bacterial ASVs showing significant disease association in at least one cohort. For each of those ASVs, the direction of change and the effect size was calculated, labeled herein as normalized rank mean difference (NRMD) between cases as controls, in each disease-cohort but now including all samples in each case-controls comparison across all studies (see methods). The heatmap in FIG. 2B highlights all those 731 ASVs, where blue indicates negative effect size (lower NRMD between cases and controls) and red indicates positive effect size (higher NRMD between cases and controls) in the indicated disease state.

### Example 2: Similarities and Differences in the microbial composition Within and Between Disease States

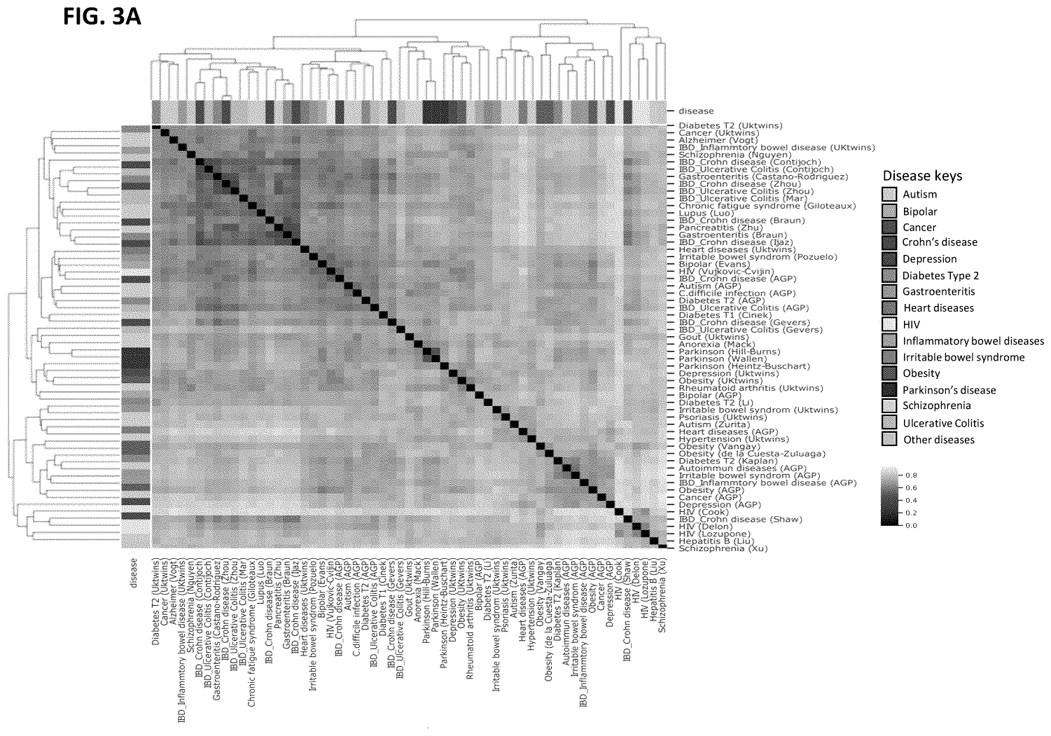

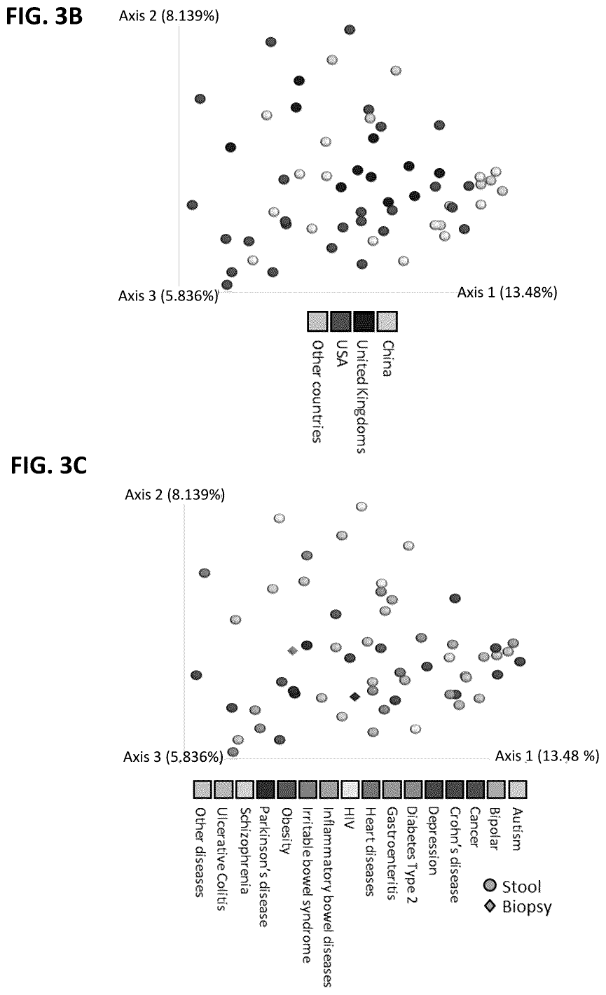

To evaluate the ability of the pipeline to reduce the cohort specific contribution and to capture signals across diseases and studies, a Bray Curtis based Principal Coordinates Analysis (PCoA) was used either on the actual mean ASVs relative abundance in cases and controls in each study (FIGS. 1C-1D), or on the NRMD effect size resulting from the pipeline. The full NRMD distance matrix between disease-cohort' effect size (FIG. 3A) showed distances cluster according to disease rather than study. Unlike using the actual mean relative abundance in cases and controls in each study, where diseases and controls from each cohort tended to be positioned in a specific area (FIG. 1C), by analyzing and plotting the effect size (NRMD), it was possible to eliminate much of the cohort specific signal, with the signal from the different cohorts being spread-out (FIG. 1D). Additional coloring of the PCoA based on the effect size by country of origin, and disease (FIG. 3) showed that cohorts from the same geographic region are spread through the graph (FIG. 3B) and there are some diseases, such as IBD and Parkinson that tended to cluster together (FIG. 3C).

The NRMD based distance matrix showed similarities between disease-cohort systematically across comparisons and is shown as a heatmap, where disease cohorts that show higher similarities are clustered together. For example, three Parkinson studies, originating from 3 different cohorts (2 from the US and 1 from Europe) showed high similarities with each other (FIG. 3A). IBD studies were significantly enriched in the left main dendrogram branch (Chi squares p value=0.009), where 11/28 (39%) IBD studies clustered on left branch vs. 3/30 (10%) IBD studies that clustered on the right branch. However, 17 other studies including diabetes type 2 (T2), Alzheimer, Lupus, heart disease, and autism also clustered on the left dendrogram branch, indicating that while there is a microbial alteration that can be detected and enriched in most of the IBD studies, this signal is not IBD-specific and a similar signal can also be detected in other diseases.

### Example 3: Non-Specific General Disease Microbial Composition

Despite some similarities in the microbial composition between datasets for the same disease (FIG. 3), similarities and clustering of different diseases were also observed, unexpectedly indicating that some of the microbial alterations may be more general and shared across several pathogenic conditions and are non-specific to a specific disease type. To define those surprising non-specific general changes across different diseases, ASVs whose NRMD effect size direction (i.e., higher in cases or controls) significantly differed from a 0.5/0.5 binomial distribution were searched for. Advantageously, this resulted in 128 ASVs that have similar behavior across multiple disease, and therefore associated with general disease state. Most (97 ASVs) showed reduced abundance shared across different diseases, and only 31 showed higher abundance across different diseases (FIG. 4A). The relative taxonomy composition of the two groups shows Bacteroidetes comprise 11 of the 97 nonspecific health-associated ASVs, whereas none were within the 31 disease-associated ASVs (Chi Square p=0.03, FIG. 4B). In contrast, Actinobacteria taxa (Chi Square p=0.09) were more abundant within disease associated ASVs (3/31) compared to health-associated ASVs (1/97) (FIG. 4B). Lactobacillales order (Firmicutes phyla) were also significantly enriched within the disease-associated ASVs (5/31 vs. 0/97 for disease-and health-associated ASVs respectively, Chi Square p<0.001). Looking into terms associated with those 31 disease-associated ASVs using dbBact (http://dbbact.org/), advantageously indicated that those tended to be associated with in feces and adult humans, and lower in control and rural communities (FIG. 4D, see methods for details).

To infer microbial functions enriched in the health vs. disease-associated bacteria, Picrust2 was applied. Using per-ASV prediction of KEGG ontologies, KEGG ontologies which are significantly more common in the health or disease-associated ASVs were searched for. 23 pathways were identified, 9 that were more common in disease and 14 more common in controls (rank-mean test with dsFDR multiple hypothesis correction FDR<0.1, FIG. 4C). Pathways more common in disease included carbohydrate metabolism, whereas pathways more common in controls included metabolism of cofactors and vitamins, and amino acid metabolism. Interestingly, cellular community pathways were more common in disease-associated ASVs, and those were more specifically associated with quorum sensing genes, a process allowing bacteria populations to communicate and coordinate group behavior and is more commonly used by pathogens.

### Example 4: IBD-Specific Microbial Alteration is Predominantly Linked to Increased Abundance of Individual Taxa

Next, ASVs specifically associated with CD/UC were defined. For that, the naive approach of just performing differential abundance on CD/UC samples compared to controls without taking into account other disease conditions does not suffice, since many of the ASVs are also associated with other diseases and likely represent general disease signal rather than uniquely related to IBD phenotypes. Therefore, there was a need to account for and use all other disease-cohorts results, to specifically identify ASVs that show more substantial and significant differences than in other diseases. CD/UC-specific ASVs were defined as ASVs showing significantly higher or lower NRMD effect size in fecal samples from CD and UC disease-cohorts (n=10) in comparison to other non-IBD disease-cohorts (n=45) (using permutation-based rank mean test of the per-disease-cohort NRMDs, with dsFDR=0.1 multiple hypothesis correction). 15 ASVs were significantly related to UC and CD, with 13 showing a higher NRMD between UC and CD cases and controls, and 2 showing a decrease NRMD between UC/CD and controls (FIG. 5A). Those specific CD and UC enriched ASVs included taxa form Gemellaceae, Veillonellaceae, Neisseriaceae and Streptococcaceae families. Term enrichments of those 13 CD/UC-associated specific ASVs using dbbact database, showed enrichment for microbial taxa seen in saliva and taxa lower in controls. (FIG. 5B). Attempts to find other disease-specific signals, including for Parkinson's disease failed, likely to lack of sufficient studies linked with a specific condition (e.g., analysis is performed at the study level, and there are only 3 Parkinson's studies analyzed).

### Example 5: A Disease Classifier Accurately Differentiating Between Many Sick vs. Healthy State Based on Non-Specific Shared Microbial Disease Signal

A common use of identifying microbial changes associated with a given disease is the advantageous creation of a machine learning classifier to differentiate between healthy controls and disease cases. However, the fact that a large set of bacteria display a consistent change across multiple disease raises the concern that classifiers may be using this unexpected, shared signal as part of the case/control classification, a fact that may lead to incorrect disease identification, and the inability to differentiate between diseases. To test this, a supervised learning Random Forests (RF) was used. For each disease-cohort, a random forest classifier was separately trained to differentiate cases/controls for this disease cohort, and this classifier was then tested on its ability to differentiate cases/controls on a different disease cohort (see methods). Additionally, for each disease cohort the performance of the classifier was tested within the disease cohort itself using a 2:1 training/validation split. AUC performance of the inter and intra-disease classification are shown in FIG. 6A, where each row represents a disease cohort on which a classifier was trained, columns represent the disease cohort on which the classifier was tested. As an estimate for the inherent noise present in the classifier performance, the performance of the same procedure was also tested where the case/control labels of each testing disease cohort were randomly shuffled (FIG. 6B). Predicting case/control state in UC and CD by using models built upon other UC and CD cohorts worked relatively well and mostly above what is expected by random. However, models built on other diseases also predicted relatively well CD and UC, and vice-versa, models built on CD and UC were able to predict other diseases. Those predications were far above the randomly mixed case-controls samples (FIG. 6B and FIG. 7A and FIG. 7B) indicating they do not occur by chance. Advantageously, those results indicate that disease classifier performs well in identifying many sick vs. healthy state based on the non-specific shared microbial disease signal. The classifier differentiate between different diseases less optimally. This advantageous ability of the classifier to separate between disease cases and controls across cohorts and diseases, substantial better than random, indicates that the disease-associated signal is surprisingly strong and consistent and can be traced despite studies-specific processing or geography.

### Example 6: Dysbiosis Index and Dysbiosis Index Score

The aim was to generate a non-specific dysbiosis index (NSDI) across different diseases, that will uniformly capture this non-specific signal, and advantageously identify the general disease-associated microbial alteration. The NSDI is built on the identified 128 non-specific ASVs. The per-sample NSDI was calculated by rank-transforming the bacteria within the sample and computing the normalized log ratio of the sum of the ranks of the 97 health-associated and 31 disease-associated ASVs. Then, the NSDI was compared to the previously published taxonomy-based CD dysbiosis index. The resulting NSDI that advantageously recapitulates a consistent change that persists across multiple diseases performed better than the previously published CD dysbiosis index, indicating that NSDI can successfully differentiate between most cases and controls across a wide variety of diseases (FIG. 6C and FIG. 8). Among the UC/CD studies, NSDI showed more significant changes between cases and controls (11/14 studies) than CD dysbiosis index (7/14). Similar results were shown in other diseases such as Parkinson's; NSDI succeeded to show significant changes in dysbiosis index in 2 out of 3 studies included in the metanalysis, in comparison to 0 out of 3 in the CD dysbiosis index. These results indicate that NSDI can successfully differentiate between most cases and controls across a wide variety of diseases.

The herein disclosed non-specific dysbiosis index (NSDI) and machine learning based classifier (Examples 5 and 6, respectively) exemplify the advantage of the herein disclosed process/method for computing a dysbiosis index score (“DI score”), that relies on large datasets which recapitulate a consistent change that persists across multiple diseases, in order to differentiate between healthy controls and disease cases across a plurality of diseases, thereby assessing/classifying the health status associated microbiome profile based on the DI score.

### Example 7: NSDI Show Differences Between Cases and Controls in disease Other Than Those Used to Generate the NSDI

The NSDI was further evaluated for its ability to differentiate between cases and controls in diseases other than those used to generate the NSDI. FIG. 10 presents NSDI performance and shows the differences between cases and controls of samples obtained from lung cancer patients (FIG. 10B) and subjects hospitalized in rehab (FIG. 10C). The results indicate that NSDI advantageously outperforms the alpha diversity score in differentiating cases and controls (FIG. 10B compared with FIG. 10A) and generally indicate on NSDI ability to significantly differentiate between cases and control in diseases other than those used to generate the NSDI.

1. A computer-implemented method for assessing/classifying a health status associated microbiome profile of a gastrointestinal (GI) sample, the method comprising:
receive data regarding an expression level of at least about 40 non-specific amplicon sequence variants (ASVs) of a V4 region of 16S rRNA in a GI sample of a subject;
computing a dysbiosis index (DI) score for the GI sample by applying a trained machine learning algorithm on the expression level of the at least about 40 ASVs, wherein an altered expression of at least a portion of the at least about 40 ASVs is shared by a plurality of diseases, and wherein at least 5 of the non-specific ASVs have a sequence as set forth in any one of SEQ ID NO: 1-42;
assessing/classifying the health status associated microbiome profile of the subject based on the DI score.

2. The method of claim 1, wherein the machine learning algorithm is trained on a data set comprising the expression level of the at least about 40 ASVs of a large plurality of GI samples obtained from subjects suffering from one or more of the plurality of diseases and from healthy subjects, and a plurality of labels associated with the large plurality of GI samples, each label indicating the health status of the GI sample.

3. The method of claim 1, wherein said altered expression of ASVs of the V4 region of 16S rRNA comprises comparing the GI sample to control samples.

4. The method of claim 1, wherein the altered expression of the at least portion of the at least about 40 ASVs comprises upregulation in the expression of about 15 ASVs and downregulation in the expression of about 5 ASVs.

5. The method of claim 4, wherein the altered expression of the at least portion of the at least about 40 ASVs further comprises upregulation in the expression of about 30 ASVs and downregulation in the expression of about 10 ASVs.

6. The method of claim 1, wherein said assessing a health status associated microbiome profile of a gastrointestinal (GI) sample comprises identifying dysbiosis in a GI sample.

7. The method of claim 6, wherein said identifying dysbiosis in a GI sample comprises prioritizing the degree of dysbiosis.

8. The method of claim 7, wherein said prioritizing the degree of dysbiosis in a GI sample comprises computing a dysbiosis index (DI) score.

9. (canceled)

10. The method of claim 1, wherein the machine learning algorithm comprises a classifier that implements disease status classification of the GI sample to a plurality of disease categories selected from inflammatory diseases, autoimmune diseases, infectious diseases, psychiatric diseases, neurological disorders, metabolic diseases, inflammatory bowel diseases, malignancies, and any combination thereof.

11. The method of claim 1, wherein the machine learning algorithm comprises a classifier that further implements disease status classification of the GI sample to a plurality of diseases selected from a group of GI diseases consisting of: crohn's disease, ulcerative colitis, inflammatory bowel disease, irritable bowel disease, gastroenteritis, clostridioides difficile infection, and cancer, and/or selected from a group of non-GI diseases consisting of: Alzheimer, anorexia, autism, bipolar, depression, chronic fatigue syndrome, diabetes T1, diabetes T2, gout, heart disease, HIV, hepatitis, hypertension, lupus, obesity, pancreatitis, rheumatoid arthritis, schizophrenia, parkinson's disease, and psoriasis.

12. The method of claim 1, further comprising identifying one or more etiologies associated with the GI sample, and wherein identifying the one or more etiologies associated with the GI sample comprises further analyzing the expression level of disease-specific ASVs of the V4 region of 16S rRNA.

13. The method of claim 12, further comprising classifying the subject as suffering from one/or more diseases based on the identified one or more etiologies associated with the GI sample.

14. (canceled)

15. The method of claim 13, wherein the analysis of altered expression of disease-specific ASVs comprises about 15 IBD-specific ASVs.

16. The method of claim 15, wherein an altered expression of at least a portion of the IBD-specific ASVs is shared by Ulcerative colitis (UC)- and Crohn's disease (CD) and wherein at least 3 of the IBD-specific ASVs have a sequence as set forth in any one of SEQ ID NO: 129-143.

17. The method of claim 15, wherein the altered expression of the about 15 IBD-specific ASVs comprises upregulation in the expression of about 13 ASVs and downregulation in the expression of about 2 ASVs.

18. The method of claim 13, wherein the one or more etiologies related to the IBD-specific ASVs are selected from Ulcerative colitis (UC) and Crohn's disease (CD).

19. The method of claim 1, further comprising the step of prioritizing and/or selecting a donor sample for fecal microbiota transplantation (FMT), wherein the sample has a low disease probability.

20. (canceled)

21. A kit comprising: primers capable of identifying at least about 40 non-specific amplicon sequence variants (ASVs) of the V4 region of 16S rRNA in a GI sample of a subject.

22. The kit of claim 21, wherein at least 5 of the non-specific ASVs identified by the primers are selected from SEQ ID NO: 1-42, and wherein said primers comprise at least a single pair of primers and wherein each pair can identify at least a single ASV.

23. (canceled)

24. The kit of claim 21, further comprising equipment for collecting, storing, and labeling a subject's GI sample.

25. (canceled)


SEQUENCE LISTING (143 sequences)

SEQ ID NO: 1 - DNA, 150 bp, unidentified
  source 1..150: mol_type = genomic DNA; note = SEQ ID NO: 1-143 are bacterial derived Homo Sapiens microbiome
tacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatg

SEQ ID NO: 2 - DNA, 150 bp, unidentified
  source 1..150: mol_type = genomic DNA
tacgtatggtgcaagcgttatccggatttactgggtgtaaagggagcgtagacggataggcaagtctggagtgaaaacccagggctcaaccctgggactgctttggaaactgcagatctggagtgccggagaggtaagcggaattcctag

SEQ ID NO: 3 - DNA, 150 bp, unidentified
  source 1..150: mol_type = genomic DNA
tacgtagggagcgagcgttgtccggatttactgggtgtaaagggtgcgtaggcggatctgcaagtcagtagtgaaatcccagggcttaaccctggaactgctattgaaactgtgggtcttgagtgaggtagaggcaggcggaattcccgg

SEQ ID NO: 4 - DNA, 150 bp, unidentified
  source 1..150: mol_type = genomic DNA
tacgtaggtcccgagcgttgtccggatttattgggcgtaaagggagcgcaggcggtcaggaaagtctggagtaaaaggctatggctcaaccatagtgtgctctggaaactgtctgacttgagtgcagaaggggagagtggaattccatgt

SEQ ID NO: 5 - DNA, 150 bp, unidentified
  source 1..150: mol_type = genomic DNA
tacgtagggggcgagcgttatccggattcattgggcgtaaagcgcgcgtaggcggcgcgccaagcggaacctctaacctcggggctcaacctcgagccgggttccgaactggcgcgctcgagttcggtagaggcaggcggaattcccggt

SEQ ID NO: 6 - DNA, 150 bp, unidentified
  source 1..150: mol_type = genomic DNA
tacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctag

SEQ ID NO: 7 - DNA, 150 bp, unidentified
  source 1..150: mol_type = genomic DNA
aacgtagggtgcaagcgttgtccggaattactgggtgtaaagggagcgcaggcggattggcaagttgggagtgaaatctatgggctcaacccataaattgctttcaaaactgtcagtcttgagtggtgtagaggtaggcggaattcccgg

SEQ ID NO: 8 - DNA, 150 bp, unidentified
  source 1..150: mol_type = genomic DNA
tacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctag

SEQ ID NO: 9 - DNA, 150 bp, unidentified
  source 1..150: mol_type = genomic DNA
tacgtatggtgcaagcgttatccggatttactgggtgtaaagggagcgtagacggttgtgtaagtctgatgtgaaagcccggggctcaaccccgggactgcattggaaactatgtaactagagtgtcggagaggtaagcggaattcctag

SEQ ID NO: 10 - DNA, 150 bp, unidentified
  source 1..150: mol_type = genomic DNA
tacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatg

SEQ ID NO: 11 - DNA, 150 bp, unidentified
  source 1..150: mol_type = genomic DNA
tacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtg

SEQ ID NO: 12 - DNA, 150 bp, unidentified
  source 1..150: mol_type = genomic DNA
tacgtagggggcaagcgttatccggatttactgggtgtaaagggagcgtagacggcatggcaagtctgaagtgaaaacccagggctcaaccctgggactgctttggaaactgtcaagctagagtgcaggagaggtaagtggaattcctag

SEQ ID NO: 13 - DNA, 150 bp, unidentified
  source 1..150: mol_type = genomic DNA
tacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatg

SEQ ID NO: 14 - DNA, 150 bp, unidentified
  source 1..150: mol_type = genomic DNA
tacgtagggggcaagcgttatccggatttactgggtgtaaagggagcgtagacggcatggcaagtctgaagtgaaagcccggggctcaaccccgggactgctttggaaactgtcaggctagagtgctggagaggtaagtggaattcctag

SEQ ID NO: 15 - DNA, 150 bp, unidentified
  source 1..150: mol_type = genomic DNA
tacgtagggggcaagcgttatccggatttactgggtgtaaagggagcgtagacggagcagcaagtctgatgtgaaaggcgggggctcaacccccggactgcattggaaactgttgatcttgagtaccggagaggtaagcggaattcctag

SEQ ID NO: 16 - DNA, 150 bp, unidentified
  source 1..150: mol_type = genomic DNA
tacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctag

SEQ ID NO: 17 - DNA, 150 bp, unidentified
  source 1..150: mol_type = genomic DNA
tacgtagggagcaagcgttgtccggatttactgggtgtaaagggtgcgtaggcggtttggtaagtcagaagtgaaatccatgggcttaacccatgaactgcttttgaaactatcgaacttgagtgaagtagaggtaggcggaattcccgg

SEQ ID NO: 18 - DNA, 150 bp, unidentified
  source 1..150: mol_type = genomic DNA
tacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctag

SEQ ID NO: 19 - DNA, 150 bp, unidentified
  source 1..150: mol_type = genomic DNA
tacgtagggggcaagcgttatccggatttactgggtgtaaagggagcgtagacggcatgacaagccagatgtgaaaacccagggctcaaccctgggactgcatttggaactgccaggctggagtgcaggagaggtaagcggaattcctag

SEQ ID NO: 20 - DNA, 150 bp, unidentified
  source 1..150: mol_type = genomic DNA
tacgtagggcgcgagcgttgtccggaattattgggcgtaaagagcttgtaggcggtttgtcgcgtctgctgtgaaaggccggggcttaaccccgtgtattgcagtgggtacgggcagactagagtgcagtaggggagactggaattcctg

SEQ ID NO: 21 - DNA, 150 bp, unidentified
  source 1..150: mol_type = genomic DNA
tacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctag

SEQ ID NO: 22 - DNA, 150 bp, unidentified
  source 1..150: mol_type = genomic DNA
tacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggt

SEQ ID NO: 23 - DNA, 150 bp, unidentified
  source 1..150: mol_type = genomic DNA
tacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctag

SEQ ID NO: 24 - DNA, 150 bp, unidentified
  source 1..150: mol_type = genomic DNA
tacgtagggggcaagcgttatccggatttactgggtgtaaagggagcgtagacggcgacgcaagtctggagtgaaagcccggggcccaaccccgggactgctttggaaactgtgctgctggagtgcaggagaggtaagtggaattcctag

SEQ ID NO: 25 - DNA, 150 bp, unidentified
  source 1..150: mol_type = genomic DNA
tacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgt

SEQ ID NO: 26 - DNA, 150 bp, unidentified
  source 1..150: mol_type = genomic DNA
tacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatg

SEQ ID NO: 27 - DNA, 150 bp, unidentified
  source 1..150: mol_type = genomic DNA
tacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctag

SEQ ID NO: 28 - DNA, 150 bp, unidentified
  source 1..150: mol_type = genomic DNA
tacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctag

SEQ ID NO: 29 - DNA, 150 bp, unidentified
  source 1..150: mol_type = genomic DNA
tacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctag

SEQ ID NO: 30 - DNA, 150 bp, unidentified
  source 1..150: mol_type = genomic DNA
tacgtaggtggcgagcgttatccggatttactgggcgtaaagggagcgtaggcggatgattaagtgggatgtgaaatacccgggctcaacttgggtgctgcattccaaactggttatctagagtgcaggagaggagagtggaattcctag

SEQ ID NO: 31 - DNA, 150 bp, unidentified
  source 1..150: mol_type = genomic DNA
tacgggggatgcgagcgttatccggatttattgggtttaaagggcgcgtaggcgggacgccaagtcagcggtaaaagactgcagctaaactgtagcacgccgttgaaactggcgccctcgagacgagacgagggaggcggaacaagtgaa

SEQ ID NO: 32 - DNA, 150 bp, unidentified
  source 1..150: mol_type = genomic DNA
tacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacg

SEQ ID NO: 33 - DNA, 150 bp, unidentified
  source 1..150: mol_type = genomic DNA
tacgtaggtggcgagcgttatccggaattactgggtgtaaagggtgtgtaggcggggaagcaagtcagatgtgaaaaccaaaggctcaacctttggcttgcatttgaaactgtttttcttgagagtgggagaggtaaacggaattcctag

SEQ ID NO: 34 - DNA, 150 bp, unidentified
  source 1..150: mol_type = genomic DNA
tacgtagggtgcaagcgttaatcggaattactgggcgtaaagcgtgcgcaggcggttctgtaagatagatgtgaaatccccgggctcaacctgggaattgcatatatgactgcaggacttgagtttgtcagaggagggtggaattccacg

SEQ ID NO: 35 - DNA, 150 bp, unidentified
  source 1..150: mol_type = genomic DNA
tacgtagggggcaagcgttatccggaattactgggtgtaaagggagcgtaggcggcatggtaagtaagatgtgaaagcccgaggcttaacctcgaggattgcattttaaactatcaagctagagtacaggagaggaaagcggaattccta

SEQ ID NO: 36 - DNA, 150 bp, unidentified
  source 1..150: mol_type = genomic DNA
tacgtagggagcaagcgttatccggatttattgggtgtaaagggtgcgtagacgggaaattaagttagttgtgaaagccctcggcttaactgaggaactgcaactaaaactggttttcttgagtgcaggagaggtaagtggaattcctag

SEQ ID NO: 37 - DNA, 150 bp, unidentified
  source 1..150: mol_type = genomic DNA
tacgtagggagcaagcgttatccggatttattgggtgtaaagggtgcgtagacgggagattaagttagttgtgaaagccctcggcttaactgaggaattgcaactaaaactggttttcttgagtgcaggagaggtaagtggaattcctag

SEQ ID NO: 38 - DNA, 150 bp, unidentified
  source 1..150: mol_type = genomic DNA
tacgtatggggcgagcgttatccggattcattgggcgtaaagcgcgcgtaggcggagcgctaagcgggacctctaacccgagggctcaacccccggccgggtcccgaactggcgctctcgagtgcggtaggggagagcggaattcccggt

SEQ ID NO: 39 - DNA, 150 bp, unidentified
  source 1..150: mol_type = genomic DNA
tacgggggatgcgagcgttatccggatttattgggtttaaagggcgcgtaggcgggacgacaagtcagcggtaaaagactgcagctaaactgtagcacgccgttgaaactgtcgacctggagacgagacgagggaggcggaacaagtgaa

SEQ ID NO: 40 - DNA, 150 bp, unidentified
  source 1..150: mol_type = genomic DNA
tacgtaggtggcaagcgttgtccggatttactgggtgtaaagggcgtgtaggcggggctgcaagtcagaagtgaaatccatgggcttaacccatgaactgcttttgaaactgtggctcttgagtatcggagaggcaggcggaattcctag

SEQ ID NO: 41 - DNA, 150 bp, unidentified
  source 1..150: mol_type = genomic DNA
tacggaaggtccgggcgttatccggatttattgggtttaaagggagcgtaggccgtttggtaagcgtgttgtgaaatgtcggggctcaacctgggcattgcagcgcgaactgccagacttgagtgcgcaggaagtaggcggaattcgtcg

SEQ ID NO: 42 - DNA, 150 bp, unidentified
  source 1..150: mol_type = genomic DNA
tacgtaggttgcaagcgttgtccggatttactgggtgtaaagggcgtgtaggcggagatgcaagttaggagtgaaatctatgggctcaacccataaactgcttctaaaactgtatcccttgagtatcggagaggcaagcggaattcctag

SEQ ID NO: 43 - DNA, 150 bp, unidentified
  source 1..150: mol_type = genomic DNA
tacgtaggtggcaagcgttgtccggaattattgggcgtaaagcgcgcgcaggcggataggtcagtctgtcttaaaagttcggggcttaaccccgtgatgggatggaaactgccaatctagagtatcggagaggaaagtggaattcctagt

SEQ ID NO: 44 - DNA, 150 bp, unidentified
  source 1..150: mol_type = genomic DNA
aacatagggggcaagcgttgtccggaatcactgggcgtaaagggcgcgtaggtggtctgttaagtcagatgtgaaatgtaagggctcaacccttaacgtgcatctgatactggcagacttgagtgcggaagaggcaagtggaattcctag

SEQ ID NO: 45 - DNA, 150 bp, unidentified
  source 1..150: mol_type = genomic DNA
aacgtagggtgcaagcgttgtccggaattactgggtgtaaagggagcgcaggcgggaagacaagttggaagtgaaaaccatgggctcaacccatgaattgctttcaaaactgtttttcttgagtagtgcagaggtagatggaattcccgg

SEQ ID NO: 46 - DNA, 150 bp, unidentified
  source 1..150: mol_type = genomic DNA
aacgtagggtgctagcgttgtccggaattactgggtgtaaagggagcgcaggtggcagtgcaagttggatgtaaaatctacgggctcaacccgtataagcattcaaaactgtgcagcttgagtgaagtagaggtaggtggaatttccggt

SEQ ID NO: 47 - DNA, 150 bp, unidentified
  source 1..150: mol_type = genomic DNA
aacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccgg

SEQ ID NO: 48 - DNA, 150 bp, unidentified
  source 1..150: mol_type = genomic DNA
aacgtaggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccgg

SEQ ID NO: 49 - DNA, 150 bp, unidentified
  source 1..150: mol_type = genomic DNA
aacgtaggtcacaagcgttgtccggaattactgggtgtaaagggagcgcaggcgggcgatcaagttggaagtgaaatccatgggctcaacccatgaactgctttcaaaactggtcgtcttgagtagtgcagaggtaggcggaattcccgg

SEQ ID NO: 50 - DNA, 150 bp, unidentified
  source 1..150: mol_type = genomic DNA
tacggaggatccaagcgttatccggatttattgggtttaaagggtgcgtaggcggtttagtaagtcagcggtgaaattttggtgcttaacaccaaacgtgccgttgatactgctgggctagagagtagttgcggtaggcggaatgtatgg

SEQ ID NO: 51 - DNA, 150 bp, unidentified
  source 1..150: mol_type = genomic DNA
tacggaggatccgagcgttatccggatttattgggtttaaagggagcgtagatgggttgttaagtcagttgtgaaagtttgcggctcaaccgtaaaattgcaattgatactggcagtcttgagtacagttgaggtaggcggaattcgtgg

SEQ ID NO: 52 - DNA, 150 bp, unidentified
  source 1..150: mol_type = genomic DNA
tacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtgg

SEQ ID NO: 53 - DNA, 150 bp, unidentified
  source 1..150: mol_type = genomic DNA
tacggaggatccgagcgttatccggatttattgggtttaaagggagcgtaggcgggtgcttaagtcagttgtgaaagtttgcggctcaaccgtaaaattgcagttgatactgggtaccttgagtgcagcataggtaggcggaattcgtgg

SEQ ID NO: 54 - DNA, 150 bp, unidentified
  source 1..150: mol_type = genomic DNA
tacggaggatgcgagcgttatccggatttattgggtttaaagggagcgcagacgggagattaagtcagttgtgaaagtttgcggctcaaccgtaaaattgcagttgatactggtttccttgagtgcagttgaggcaggcggaattcgtgg

SEQ ID NO: 55 - DNA, 150 bp, unidentified
  source 1..150: mol_type = genomic DNA
tacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtgg

SEQ ID NO: 56 - DNA, 150 bp, unidentified
  source 1..150: mol_type = genomic DNA
tacggaggattcaagcgttatccggatttattgggtttaaagggtgcgtaggcggtttgataagttagaggtgaaatgttagggctcaaccctgaaactgcctctaatactgttggactagagagtagttgcggtaggcggaatgtatgg

SEQ ID NO: 57 - DNA, 150 bp, unidentified
  source 1..150: mol_type = genomic DNA
tacggaggattcgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtgg

SEQ ID NO: 58 - DNA, 150 bp, unidentified
  source 1..150: mol_type = genomic DNA
tacgtagggagcaagcgttatccggatttattgggtgtaaagggtgcgtagacgggaaggtaagttagttgtgaaatccctcggctcaactgaggaactgcgactaaaactgcttttcttgagtgctggagaggaaagtggaattcctag

SEQ ID NO: 59 - DNA, 150 bp, unidentified
  source 1..150: mol_type = genomic DNA
tacgtagggagcaagcgttatccggatttattgggtgtaaagggtgcgtagacgggacaacaagttagttgtgaaatccctcggcttaactgaggaactgcaactaaaactattgttcttgagtgttggagaggaaagtggaattcctag

SEQ ID NO: 60 - DNA, 150 bp, unidentified
  source 1..150: mol_type = genomic DNA
tacgtagggagcaagcgttgtccggatttactgggtgtaaagggtgcgtaggcggctttgcaagtcagatgtgaaatctatgggctcaacccataaactgcatttgaaactgtagagcttgagtgaagtagaggcaggcggaattccccg

SEQ ID NO: 61 - DNA, 150 bp, unidentified
  source 1..150: mol_type = genomic DNA
tacgtagggagcgagcgttgtccggaattactgggtgtaaagggagcgtaggcgggaaagcaagttggaagtgaaatgcatgggcttaacccatgagctgctttcaaaactgtttttcttgagtgaagtagaggcaggcggaattcctag

SEQ ID NO: 62 - DNA, 150 bp, unidentified
  source 1..150: mol_type = genomic DNA
tacgtagggagcgagcgttgtccggaattactgggtgtaaagggagcgtaggcgggatcgcaagtcagatgtgaaaactatgggcttaacccataaactgcatttgaaactgtggttcttgagtgaagtagaggtaagcggaattcctag

SEQ ID NO: 63 - DNA, 150 bp, unidentified
  source 1..150: mol_type = genomic DNA
tacgtagggagcgagcgttgtccggaattactgggtgtaaagggagcgtaggcgggatcttaagtcaggtgtgaaaactatgggctcaacccatagactgcacttgaaactgaggttcttgagtgaagtagaggcaggcggaattcctag

SEQ ID NO: 64 - DNA, 150 bp, unidentified
  source 1..150: mol_type = genomic DNA
tacgtagggggcaagcgttatccggaattactgggtgtaaagggtgagtaggcggcatgacaagtaagatgtgaaagcccgtggcttaaccacgggattgcattttaaactgttgagctagagtacaggagaggaaagcggaattcccag

SEQ ID NO: 65 - DNA, 150 bp, unidentified
  source 1..150: mol_type = genomic DNA
tacgtagggggcaagcgttatccggatttactgggtgtaaagggagcgcagacggcactgcaagtctgaagtgaaagcccggggctcaaccccgggactgctttggaaactgtagagctagagtgctggagaggcaagcggaattcctag

SEQ ID NO: 66 - DNA, 150 bp, unidentified
  source 1..150: mol_type = genomic DNA
tacgtagggggcaagcgttatccggatttactgggtgtaaagggagcgcaggcggtctggcaagtctgatgtgaaatcccggggctcaactccggaattgcattggaaactgtcagactagagtgccggagaggtaagtggaattcctag

SEQ ID NO: 67 - DNA, 150 bp, unidentified
  source 1..150: mol_type = genomic DNA
tacgtagggggcaagcgttatccggatttactgggtgtaaagggagcgtagacggaagagcaagtctgatgtgaaaggctggggcttaaccccaggactgcattggaaactgtttttcttgagtgccggagaggtaagcggaattcctag

SEQ ID NO: 68 - DNA, 150 bp, unidentified
  source 1..150: mol_type = genomic DNA
tacgtagggggcaagcgttatccggatttactgggtgtaaagggagcgtagacggaatggcaagtctgatgtgaaaggccggggctcaaccccgggactgcattggaaactgtcaatctagagtaccggaggggtaagtggaattcctag

SEQ ID NO: 69 - DNA, 150 bp, unidentified
  source 1..150: mol_type = genomic DNA
tacgtagggggcaagcgttatccggatttactgggtgtaaagggagcgtagacggatggacaagtctgatgtgaaaggctggggctcaaccccgggactgcattggaaactgcccgtcttgagtgccggagaggtaagcggaattcctag

SEQ ID NO: 70 - DNA, 150 bp, unidentified
  source 1..150: mol_type = genomic DNA
tacgtagggggcaagcgttatccggatttactgggtgtaaagggagcgtagacggcaaggcaagtctgaagtgaaagcccggtgcttaacgccgggactgctttggaaactgtttagctggagtgccggagaggtaagcggaattcctag

SEQ ID NO: 71 - DNA, 150 bp, unidentified
  source 1..150: mol_type = genomic DNA
tacgtagggggcaagcgttatccggatttactgggtgtaaagggagcgtagacggcaaggcaagtctgaagtgaaagcccggtgcttaacgccgggactgctttggaaactgtttggctggagtgccggagaggtaagcggaattcctag

SEQ ID NO: 72 - DNA, 150 bp, unidentified
  source 1..150: mol_type = genomic DNA
tacgtagggggcaagcgttatccggatttactgggtgtaaagggagcgtagacggcaaggcaagtctgatgtgaaaggctggggctcaaccccgggactgcattggaaactgtcctgctggagtgccggagaggtaagcggaattcctag

SEQ ID NO: 73 - DNA, 150 bp, unidentified
  source 1..150: mol_type = genomic DNA
tacgtagggggcaagcgttatccggatttactgggtgtaaagggagcgtagacggcagcgcaagtctgaagtgaaagcccggggctcaaccccggaatggctttggaaactgtgcagctagagtaccggaggggtaagcggaattcctag

SEQ ID NO: 74 - DNA, 150 bp, unidentified
  source 1..150: mol_type = genomic DNA
tacgtagggggcaagcgttatccggatttactgggtgtaaagggagcgtagacggcatggcaagtctgaagtgaaatgcgggggctcaacccctgaactgctttggaaactgtcaggctggagtgcaggagaggtaagtggaattcctag

SEQ ID NO: 75 - DNA, 150 bp, unidentified
  source 1..150: mol_type = genomic DNA
tacgtagggggcaagcgttatccggatttactgggtgtaaagggagcgtagacggcattgcaagccagatgtgaaagcccggggctcaaccccgggactgcatttggaactgtagagctagagtgtcggagaggcaagtggaattcctag

SEQ ID NO: 76 - DNA, 150 bp, unidentified
  source 1..150: mol_type = genomic DNA
tacgtagggggcaagcgttatccggatttactgggtgtaaagggagcgtagacggccgtgcaagtctgatgtgaaaggctggggctcaaccccgggactgcattggaaactgtatggctggagtgccggagaggtaagcggaattcctag

SEQ ID NO: 77 - DNA, 150 bp, unidentified
  source 1..150: mol_type = genomic DNA
tacgtagggggcaagcgttatccggatttactgggtgtaaagggagcgtagacggcgacgcaagtctgaagtgaaatacccgggctcaacctgggaactgctttggaaactgtgttgctagagtgctggagaggtaagcggaattcctag

SEQ ID NO: 78 - DNA, 150 bp, unidentified
  source 1..150: mol_type = genomic DNA
tacgtagggggcaagcgttatccggatttactgggtgtaaagggagcgtagacggcgacgcaagtctgaagtgaaatacccgggctcaacctgggaactgctttggaaactgtgttgctggagtgctggagaggtaagcggaattcctag

SEQ ID NO: 79 - DNA, 150 bp, unidentified
  source 1..150: mol_type = genomic DNA
tacgtagggggcaagcgttatccggatttactgggtgtaaagggagcgtagacggcgagacaagtctgaagtgaaagcccggggctcaaccccgggactgctttggaaactgccttgctagagtgctggagaggtaagtggaattcctag

SEQ ID NO: 80 - DNA, 150 bp, unidentified
  source 1..150: mol_type = genomic DNA
tacgtagggggcaagcgttatccggatttactgggtgtaaagggagcgtagacggcgaggcaagtctgatgtgaaaacccggggctcaaccccgtgactgcattggaaactgttttgcttgagtgccggagaggtaagcggaattcctag

SEQ ID NO: 81 - DNA, 150 bp, unidentified
  source 1..150: mol_type = genomic DNA
tacgtagggggcaagcgttatccggatttactgggtgtaaagggagcgtagacggcgaggcaagtctgatgtgaaagcctggggcttaaccccggaactgcattggaaactgctttgctggagtgccggagaggtaagcggaattcctag

SEQ ID NO: 82 - DNA, 150 bp, unidentified
  source 1..150: mol_type = genomic DNA
tacgtagggggcaagcgttatccggatttactgggtgtaaagggagcgtagacggcgatgcaagtctgaagtgaaatacccgggctcaacctgggaactgctttggaaactgtatggctagagtgctggagaggtaagcggaattcctag

SEQ ID NO: 83 - DNA, 150 bp, unidentified
  source 1..150: mol_type = genomic DNA
tacgtagggggcaagcgttatccggatttactgggtgtaaagggagcgtagacggctaagcaagtcagaagtgaaaggctggggctcaaccccgggactgcttttgaaactgtttggctagagtgctggagaggtaagcggaattcctag

SEQ ID NO: 84 - DNA, 150 bp, unidentified
  source 1..150: mol_type = genomic DNA
tacgtagggggcaagcgttatccggatttactgggtgtaaagggagcgtagacggctttgcaagtctgacgtgaaaatccggggctcaaccccggaactgcgttggaaactgtgaggcttgagtgccggagaggtaagcggaattcctag

SEQ ID NO: 85 - DNA, 150 bp, unidentified
  source 1..150: mol_type = genomic DNA
tacgtagggggcaagcgttatccggatttactgggtgtaaagggagcgtagacggtcaagcaagtcagaagtgaaaggctggggctcaaccccgggactgcttttgaaactgtttgactagagtgctggagaggtaagcggaattcctag

SEQ ID NO: 86 - DNA, 150 bp, unidentified
  source 1..150: mol_type = genomic DNA
tacgtagggggcaagcgttatccggatttactgggtgtaaagggagcgtagacggtcaagcaagtcagaagtgaaaggctggggctcaaccccgggactgcttttgaaactgtttgactggagtgctggagaggtaagcggaattcctag

SEQ ID NO: 87 - DNA, 150 bp, unidentified
  source 1..150: mol_type = genomic DNA
tacgtagggggcaagcgttatccggatttactgggtgtaaagggagcgtagacggttaagcaagtcagaagtgaaaggctggggctcaaccccgggactgcttttgaaactgtttaactagagtgctggagaggtaagcggaattcctag

SEQ ID NO: 88 - DNA, 150 bp, unidentified
  source 1..150: mol_type = genomic DNA
tacgtagggggcaagcgttatccggatttactgggtgtaaagggagcgtagacggttttgcaagtctgaagtgaaagcccggggcttaaccccgggactgctttggaaactgtagaactagagtgcaggagaggtaagtggaattcctag

SEQ ID NO: 89 - DNA, 150 bp, unidentified
  source 1..150: mol_type = genomic DNA
tacgtagggggcaagcgttatccggatttactgggtgtaaagggagcgtagacggttttgcaagtctgaagtgaaagcccggggcttaaccccgggactgctttggaaactgtaggactagagtgcaggagaggtaagtggaattcctag

SEQ ID NO: 90 - DNA, 150 bp, unidentified
  source 1..150: mol_type = genomic DNA
tacgtagggggcaagcgttatccggatttactgggtgtaaagggagcgtaggcggtctgacaagtcagaagtgaaagcccggggctcaactccgggactgcttttgaaactgccggactagattgcaggagaggtaagtggaattcctag

SEQ ID NO: 91 - DNA, 150 bp, unidentified
  source 1..150: mol_type = genomic DNA
tacgtagggggcaagcgttatccggatttactgggtgtaaagggagcgtaggcggtctggcaagccagaagtgaaagcccggggcttaaccccgggactgcttttggaactgttagactagagtgtcggagaggtaagtggaattcctag

SEQ ID NO: 92 - DNA, 150 bp, unidentified
  source 1..150: mol_type = genomic DNA
tacgtagggggcaagcgttgtccggaataattgggcgtaaagggcgcgtaggcggctcggtaagtctggagtgaaagtcctgcttttaaggtgggaattgctttggatactgtcgggcttgagtgcaggagaggttagtggaattcccag

SEQ ID NO: 93 - DNA, 150 bp, unidentified
  source 1..150: mol_type = genomic DNA
tacgtagggggcgagcgttatccggatttactgggtgtaaagggagcgtagacggcgtatcaagtctgatgtgaaaggcaggggcttaacccctggactgcattggaaactggtatgcttgagtgccggaggggtaagcggaattcctag

SEQ ID NO: 94 - DNA, 150 bp, unidentified
  source 1..150: mol_type = genomic DNA
tacgtagggggcgagcgttgtccggaatcactgggcgtaaagggcgcgtaggcggcctagtaagtcagatgtgaaaagtgagggctcaacccttagagtgcatctgatactgctaggcttgagtacagaagaggaaagcggaattcctag

SEQ ID NO: 95 - DNA, 150 bp, unidentified
  source 1..150: mol_type = genomic DNA
tacgtagggggcgagcgttgtccggaatgattgggcgtaaagggcgcgtaggcggcctgctaagtctgaagtgaaagtcctgctttcaaggtgggaagtgctttggatactggtgggctggagtgcaggagaggaaagcggaattaccgg

SEQ ID NO: 96 - DNA, 150 bp, unidentified
  source 1..150: mol_type = genomic DNA
tacgtagggggcgagcgttgtccggaattactgggcgtaaagggagcgtaggcggtctgataagttggatgtgaaatacccgggcttaacttggggggtgcatccaatactgttggactagagtacaggagaggaaagcggaattcctag

SEQ ID NO: 97 - DNA, 150 bp, unidentified
  source 1..150: mol_type = genomic DNA
tacgtaggtgacaagcgttgtccggatttactgggtgtaaagggcgcgtaggcggactgtcaagtcagtcgtgaaataccggggcttaaccccggggctgcgattgaaactgacagccttgagtatcggagaggaaagcggaattcctag

SEQ ID NO: 98 - DNA, 150 bp, unidentified
  source 1..150: mol_type = genomic DNA
tacgtaggtggcaagcgttatccggatttactgggtgtaaagggcgcgtaggcggggatacaagtcagatgtgaaatctatgggcttaacccataaactgcatttgaaactgtatctcttgagtgtcggagaggtagacggaattcctag

SEQ ID NO: 99 - DNA, 150 bp, unidentified
  source 1..150: mol_type = genomic DNA
tacgtaggtggcaagcgttatccggatttactgggtgtaaagggcgtgtaggcgggaaagcaagtcagatgtgaaaactgtgggctcaacccacagcctgcatttgaaactgtttttcttgagtactggagaggcagatggaattcctag

SEQ ID NO: 100 - DNA, 150 bp, unidentified
  source 1..150: mol_type = genomic DNA
tacgtaggtggcaagcgttgtccggaattactgggtgtaaagggagcgtaggcgggatgccaagtcagctgtgaaaactatgggcttaacctgtagactgcagttgaaactggtattcttgagtgaagtagaggttggcggaattccgag

SEQ ID NO: 101 - DNA, 150 bp, unidentified
  source 1..150: mol_type = genomic DNA
tacgtaggtggcaagcgttgtccggaattactgggtgtaaagggtgcgcaggcgggattgcaagttggatgtgaaataccggggcttaaccccggagctgcatccaaaactgtagttcttgagtggagtagaggtaagcggaattccgag

SEQ ID NO: 102 - DNA, 150 bp, unidentified
  source 1..150: mol_type = genomic DNA
tacgtaggtggcaagcgttgtccggatttactgggtgtaaagggcgtgcagccgggaatgcaagtcagatgtgaaatccatgggcttaacccatgaactgcatttgaaactgtatttcttgagtactggagaggcaatcggaattcctag

SEQ ID NO: 103 - DNA, 150 bp, unidentified
  source 1..150: mol_type = genomic DNA
tacgtaggtggcaagcgttgtccggatttactgggtgtaaagggcgtgcagccgggcatgcaagtcagatgtgaaatctcagggcttaaccctgaaactgcatttgaaactgtatgtcttgagtgccggagaggtaatcggaattccttg

SEQ ID NO: 104 - DNA, 150 bp, unidentified
  source 1..150: mol_type = genomic DNA
tacgtaggtggcaagcgttgtccggatttactgggtgtaaagggcgtgcagccgggtctgcaagtcagatgtgaaatccatgggctcaacccatgaactgcatttgaaactgtagatcttgagtgtcggaggggcaatcggaattcctag

SEQ ID NO: 105 - DNA, 150 bp, unidentified
  source 1..150: mol_type = genomic DNA
tacgtaggtggcaagcgttgtccggatttactgggtgtaaagggcgtgcagccgggtgcgcaagtcagatgtgaaatctcagggctcaaccctgaaactgcatttgaaactgtgcatcttgagtgccggagaggtaatcggaattccttg

SEQ ID NO: 106 - DNA, 150 bp, unidentified
  source 1..150: mol_type = genomic DNA
tacgtaggtggcaagcgttgtccggatttactgggtgtaaagggcgtgtagccgggaaggcaagtcagatgtgaaatccacgggcttaactcgtgaactgcatttgaaactacttttcttgagtatcggagaggcaatcggaattcctag

SEQ ID NO: 107 - DNA, 150 bp, unidentified
  source 1..150: mol_type = genomic DNA
tacgtaggtggcaagcgttgtccggatttactgggtgtaaagggcgtgtagccgggagggcaagtcagatgtgaaatccacgggctcaactcgtgaactgcatttgaaactactcttcttgagtatcggagaggcaatcggaattcctag

SEQ ID NO: 108 - DNA, 150 bp, unidentified
  source 1..150: mol_type = genomic DNA
tacgtaggtggcaagcgttgtccggatttactgggtgtaaagggcgtgtagccggggagacaagtcagatgtgaaatctggaggctcaacctccaaactgcatttgaaactgtttctcttgagtatcggagaggtaatcggaattcctag

SEQ ID NO: 109 - DNA, 150 bp, unidentified
  source 1..150: mol_type = genomic DNA
tacgtaggtggcaagcgttgtccggatttactgggtgtaaagggcgtgtaggcggagatgcaagtcagatgtgaaatccccgggcttaacccgggaactgcatttgaaactgtatcccttgagtatcggagaggcaggcggaattcctag

SEQ ID NO: 110 - DNA, 150 bp, unidentified
  source 1..150: mol_type = genomic DNA
tacgtaggtggcaagcgttgtccggatttactgggtgtaaagggcgtgtaggcgggagagcaagtcagaagtgaaatctatgggcttaacccataaactgcttttgaaactgttcttcttgagtatcggagaggcaggcggaattcctag

SEQ ID NO: 111 - DNA, 150 bp, unidentified
  source 1..150: mol_type = genomic DNA
tacgtaggtggcaagcgttgtccggatttactgggtgtaaagggcgtgtaggcgggattgcaagtcaggcgtgaaaaccaggggctcaacctctggcctgcgtttgaaactgtagttcttgagtactggagaggttgacggaattcctag

SEQ ID NO: 112 - DNA, 150 bp, unidentified
  source 1..150: mol_type = genomic DNA
tacgtaggtggcgagcgttatccggaattattgggcgtaaagagggagcaggcggcactaagggtctgtggtgaaagatcgaagcttaacttcggtaagccatggaaaccgtagagctagagtgtgtgagaggatcgtggaattccatgt

SEQ ID NO: 113 - DNA, 150 bp, unidentified
  source 1..150: mol_type = genomic DNA
tacgtaggtggcgagcgttgtccggaattactgggtgtaaagggagtgtaggcgggaaggcaagtcagaagtgaaaattatgggcttaacccataacctgcttttgaaactgtttttcttgagtgaggcagaggcaagcggaattcctag

SEQ ID NO: 114 - DNA, 150 bp, unidentified
  source 1..150: mol_type = genomic DNA
tacgtaggtggcgagcgttgtccggatttactgggcgtaaagggagcgtaggcggatttttaagtgagatgtgaaatactcgggcttaacctgagtgctgcatttcaaactggaagtctagagtgcaggagaggagaagggaattcctag

SEQ ID NO: 115 - DNA, 150 bp, unidentified
  source 1..150: mol_type = genomic DNA
tacgtaggttgcaagcgttgtccggaattactgggtgtaaagggcgtgtaggcggagctgcaagtcagatgtgaaatcccggggctcaaccccggaactgcatttgaaactgtagcccttgagtatcggagaggcaagcggaattcctag

SEQ ID NO: 116 - DNA, 150 bp, unidentified
  source 1..150: mol_type = genomic DNA
tacgtatggagcaagcgttatccggatttactgggtgtaaagggagcgtaggcggtcctgcaagtctgatgtgaaaggccggggctcaaccccgggactgcattggaaactgtaggactagagtgtcggaggggtaagtggaattcctag

SEQ ID NO: 117 - DNA, 150 bp, unidentified
  source 1..150: mol_type = genomic DNA
tacgtatggagcaagcgttatccggatttactgggtgtaaagggagcgtaggcggtcttataagtctgatgtgaaagcccggggctcaaccccgggactgcattggaaactgtaggactagagtgtcggaggggtaagtggaattcctag

SEQ ID NO: 118 - DNA, 150 bp, unidentified
  source 1..150: mol_type = genomic DNA
tacgtatggagcaagcgttatccggatttactgggtgtaaagggagtgtaggtggccaggcaagtcagaagtgaaagcccggggctcaaccccgggactgcttttgaaactgcagggctagagtgcaggaggggcaagtggaattcctag

SEQ ID NO: 119 - DNA, 150 bp, unidentified
  source 1..150: mol_type = genomic DNA
tacgtatggagcaagcgttatccggatttactgggtgtaaagggagtgtaggtggccatgcaagtcagaagtgaaaatccggggctcaaccccggaactgcttttgaaactgtgaggctagagtgcaggaggggtgagtggaattcctag

SEQ ID NO: 120 - DNA, 150 bp, unidentified
  source 1..150: mol_type = genomic DNA
tacgtatggagcaagcgttatccggatttactgggtgtaaagggagtgtaggtggtatcacaagtcagaagtgaaagcccggggctcaaccccgggactgcttttgaaactgtggaactggagtgcaggagaggtaagtggaattcctag

SEQ ID NO: 121 - DNA, 150 bp, unidentified
  source 1..150: mol_type = genomic DNA
tacgtatggagcaagcgttatccggatttactgggtgtaaagggtgcgtaggtggcagtgcaagtcagatgtgaaaggccggggctcaaccccggagctgcatttgaaactgcatagctagagtacaggagaggcaggcggaattcctag

SEQ ID NO: 122 - DNA, 150 bp, unidentified
  source 1..150: mol_type = genomic DNA
tacgtatggggcaagcgttatccggatttactgggtgtaaagggagcgtaggcggcatggcaagtcagaagtgaaagcctggggctcaaccccggaattgcttttgaaactgtcaggctagagtgtcggaggggtaagcggaattcctag

SEQ ID NO: 123 - DNA, 150 bp, unidentified
  source 1..150: mol_type = genomic DNA
tacgtatggggcaagcgttatccggatttactgggtgtaaagggagcgtaggcggtaagacaagtcagaagtgaaaggctggggctcaaccctgggactgcttttgaaactgtctaactagagtgcaggagaggtaagtggaattcctag

SEQ ID NO: 124 - DNA, 150 bp, unidentified
  source 1..150: mol_type = genomic DNA
tacgtatggtgcaagcgttatccggatttactgggtgtaaagggagcgcaggcggtctggcaagtctgatgtgaaaatccggggctcaactccggaactgcattggaaactgtcagactagagtgtcggagaggtaagtggaattcctag

SEQ ID NO: 125 - DNA, 150 bp, unidentified
  source 1..150: mol_type = genomic DNA
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctag

SEQ ID NO: 126 - DNA, 150 bp, unidentified
  source 1..150: mol_type = genomic DNA
tacgtatggtgcaagcgttatccggatttactgggtgtaaagggagcgtaggtggcaaggcaagccagaagtgaaaacccggggctcaaccgcgggattgcttttggaactgtcatgctagagtgcaggaggggtgagcggaattcctag

SEQ ID NO: 127 - DNA, 150 bp, unidentified
  source 1..150: mol_type = genomic DNA
tacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctag

SEQ ID NO: 128 - DNA, 150 bp, unidentified
  source 1..150: mol_type = genomic DNA
tacgtatggtgcaagcgttatccggatttactgggtgtaaagggtgcgtaggtggtgagacaagtctgaagtgaaaatccggggcttaaccccggaactgctttggaaactgcctgactagagtacaggagaggtaagtggaattcctag

SEQ ID NO: 129 - DNA, 150 bp, unidentified
  source 1..150: mol_type = genomic DNA
tacgtagggggcaagcgttatccggatttactgggtgtaaagggagcgtaggcggccatgcaagtcagaagtgaaagcccggggctcaaccccgggactgcttttgaaactgtaaggctagattgccggagaggtaagcggaattcctag

SEQ ID NO: 130 - DNA, 150 bp, unidentified
  source 1..150: mol_type = genomic DNA
tacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagt

SEQ ID NO: 131 - DNA, 150 bp, unidentified
  source 1..150: mol_type = genomic DNA
tacgtatgtcacaagcgttatccggatttattgggcgtaaagcgcgtctaggtggttatgtaagtctgatgtgaaaatgcagggctcaactctgtattgcgttggaaactgtgtaactagagtactggagaggtaagcggaactacaagt

SEQ ID NO: 132 - DNA, 150 bp, unidentified
  source 1..150: mol_type = genomic DNA
tacgtaggtggcgagcgttgtccggatttactgggcgtaaagggagcgtaggcggatttttaagtgagatgtgaaatacccgggctcaacttgggtgctgcatttcaaactggaagtctagagtgcaggagaggagagtggaattcctag

SEQ ID NO: 133 - DNA, 150 bp, unidentified
  source 1..150: mol_type = genomic DNA
tacgtagggcgcaagcgttgtccggaattattgggcgtaaagagctcgtaggcggctggtcgcgtctgtcgtgaaaacttccggctcaaccgggggcttgcggtgggtacgggccggctagagtgcggtaggggtaactggaactcctgg

SEQ ID NO: 134 - DNA, 150 bp, unidentified
  source 1..150: mol_type = genomic DNA
tacgtagggggcaagcgttatccggatttactgggtgtaaagggagcgtagacggacgggcaagtctgatgtgaaagcccggggcttaaccccgggactgcattggaaactgtccatcttgagtgccggagaggtaagcggaattcctag

SEQ ID NO: 135 - DNA, 150 bp, unidentified
  source 1..150: mol_type = genomic DNA
tacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatg

SEQ ID NO: 136 - DNA, 150 bp, unidentified
  source 1..150: mol_type = genomic DNA
tacgtagggggctagcgttatccggatttactgggcgtaaagggtgcgtaggcggtctttcaagtcaggagtgaaaggctacggctcaaccgtagtaagctcttgaaactgggagacttgagtgcaggagaggagagtggaattcctagt

SEQ ID NO: 137 - DNA, 150 bp, unidentified
  source 1..150: mol_type = genomic DNA
tacgtaggtggcaagcgttgtccggatttattgggcgtaaagcgagcgcaggcggttccttaagtctgatgtgaaagcccccggctcaaccggggagggtcattggaaactggggaacttgagtgcagaagaggagagtggaattccatg

SEQ ID NO: 138 - DNA, 150 bp, unidentified
  source 1..150: mol_type = genomic DNA
tacgtaggtggcaagcgttgtccggaattattgggcgtaaagcgcgcgcaggcggtttcttaagtccatcttaaaagcgtggggctcaaccccatgaggggatggaaactgggaagctggagtatcggagaggaaagtggaattcctagt

SEQ ID NO: 139 - DNA, 150 bp, unidentified
  source 1..150: mol_type = genomic DNA
tacgtaggtggcaagcgttgtccggatttactgggcgtaaagggagcgtaggcggatttttaagtgggatgtgaaatacccgggctcaacctgggtgctgcattccaaactggaaatctagagtgcaggaggggaaagtggaattcctag

SEQ ID NO: 140 - DNA, 150 bp, unidentified
  source 1..150: mol_type = genomic DNA
tacgtaggtcccgagcgttgtccggatttattgggcgtaaagcgagcgcaggcggttagataagtctgaagttaaaggctgtggcttaaccatagtacgctttggaaactgtttaacttgagtgcaagaggggagagtggaattccatgt

SEQ ID NO: 141 - DNA, 150 bp, unidentified
  source 1..150: mol_type = genomic DNA
tacgtaggtggcaagcgttgtccggaattattgggcgtaaagcgcgcgcaggtggtttaataagtctgatgtgaaagcccacggctcaaccgtggagggtcattggaaactgttaaacttgagtgcaggagagaaaagtggaattcctag

SEQ ID NO: 142 - DNA, 150 bp, unidentified; identical to SEQ ID NO: 16
  source 1..150: mol_type = genomic DNA

SEQ ID NO: 143 - DNA, 150 bp, unidentified; identical to SEQ ID NO: 30
  source 1..150: mol_type = genomic DNA